(19) United States
(12) Patent Application Publication
(10) Pub. No.: US 2020/0038600 A1
(43) Pub. Date: Feb. 6, 2020

(54) DEVICE AND METHOD FOR DRUG DELIVERY

(71) Applicant: Insuline Medical Ltd., Jerusalem (IL)

(72) Inventors: Gabriel BITTON, Jerusalem (IL); Avi BEN-SIMON, Zichron-Yaakov (IL)

(21) Appl. No.: 16/339,972

(22) PCT Filed: Oct. 5, 2017
(86) PCT No.: PCT/IB2017/001363

§ 371 Date: Apr. 5, 2019

Related U.S. Application Data
(60) Provisional application No. 62/473,396, filed on Mar. 19, 2017.
(60) Provisional application No. 62/452,017, filed on Jan. 30, 2017.
(60) Provisional application No. 62/404,502, filed on Oct. 5, 2016.

(51) Int. Cl. A61M 5/44; A61M 5/142; A61M 39/02

(52) U.S. Cl. CPC A61M 5/44; A61M 2205/587; A61M 39/0208; A61M 2205/3606; A61M 2205/3673; A61M 2205/0233; A61M 2205/362; A61M 2205/3646; A61M 2230/201; A61M 2205/3633; A61M 2205/8206; A61M 2205/18; A61M 2205/502; A61M 2205/581; A61M 5/142

(57) ABSTRACT

A drug delivery control apparatus (e.g. a treatment apparatus) may be configured to control an amount of drug contained in a drug depot delivered or otherwise perfused or diffused into the circulatory system of a patient comprising a cooling element configured for cooling a treatment area by removing heat from the treatment area. The cooling element may be arranged above or near the treatment area. A heat disposal assembly is in thermal communication with the cooling element and configured for directing the removed heat to a heat zone away from the treatment area. A power source, a controller and a housing may be configured to at least partially house at least the cooling element and the heat disposal assembly.

### CROSS-REFERENCE TO RELATED APPLICATIONS

This application claims priority to U.S. Provisional Patent Application No. 62/404,502, filed Oct. 5, 2016, and entitled “Device, System and Method for Delivery of a Long-Acting Drug;” U.S. Provisional Patent Application No. 62/452,017, filed Jan. 30, 2017, and entitled “System and Method for Control of Drug Delivery;” and U.S. Provisional Patent Application No. 62/473,396, filed Mar. 19, 2017, and entitled “Device and Method for Drug Delivery,” the disclosures of which are hereby expressly incorporated by reference in their entireties.

### TECHNICAL FIELD

Embodiments of the present disclosure generally relate to systems and methods for control of drug delivery to a patient and in some cases particularly to delivery of long acting drugs.

### BACKGROUND

Drug injection by syringe, pen injectors and other devices is used regularly for subcutaneous injections of therapeutic fluids, drugs, proteins, and other compounds. Such delivery systems and methods are also used routinely for insulin delivery.

Diabetic patients may require insulin injections around the clock to maintain proper blood glucose levels. Two types of insulin drugs are usually used. The first is a long acting insulin that provides the basal insulin rate needed for maintaining patient's blood glucose level within a desired range between meals and overnight. The basal insulin may be injected at predetermined times, such as circadianly or a few times a week.

The second is a short acting insulin, bolus injection (or a “rapid-acting insulin”) that provides an amount of insulin for matching a dose of carbohydrates consumed by the patient during meals. The combination of a long acting insulin and a short acting insulin is called “basal-bolus therapy” or “intensive insulin therapy”. This therapy is used by most of diabetes mellitus type I subjects as well as by part of the diabetes mellitus type II population, which are on multiple daily insulin injection therapy. There is an additional large population of subjects, typically diabetes mellitus type II subjects, that only inject a single long acting insulin dose once a day, which needs to last the whole day.

Both long acting insulin and short acting insulin may be injected into the subcutaneous tissue at a drug depot. From the drug depot the insulin is collected by the circulatory system. Typically, for short acting insulin, concentration starts to increase approximately 10-15 minutes following injection, reaching a maximum concentration at approximately 30-60 minutes, following injection and reaching maximum effect of blood glucose levels at approximately 80-120 minutes post injection.

The insulin pharmacokinetics (PK) and pharmacodynamics (PD) temporal profile of both long acting insulin and short acting insulin can vary depending on many parameters including, for example, insulin dosage, insulin concentration, injection depth into the tissue, ambient temperature, local blood perfusion or diffusion at the injection site, exercise level, food intake, anatomical injection site in the body, such as the abdomen or buttocks, and other parameters. Variation of those parameters may result in an approximately 30%-40% or even 30%-60% or more variability in the pharmacokinetics and pharmacodynamics profiles of the injected insulin.

For long acting insulin, the requirement is that the drug concentration in the blood be as constant as possible for periods of a day or more for maintaining patient's blood glucose level within a desired, healthy range. When injecting long acting insulin, the concentration of the insulin in the blood may start to increase within a half an hour to 1-2 hours and should be constant for a period of about 24 hours. Examples of such commercially available long acting insulin analogs are insulin glargine marketed under the trade name LANTUS® and TOUJEO®, Lente insulin marketed under the trade name HUMULIN® and insulin detemir marketed under the trade name LEVEMIR®. An older version of insulin used for basal therapy is, for example, NPH (Neutral Protamine Hagedorn) insulin.

The mechanism controlling the temporal profiles of long acting insulin is different for each of the different types of insulin analogs. For example, insulin glargine is soluble at pH 4, while in neutral pH (pH 7.4) it forms precipitates. When injected into the subcutaneous tissue it resides there in the form of microprecipitates of multi-hexamer structures. The insulin glargine is slowly dissolved into single hexamers and then to dimers and/or monomers at a rate which is dependent (inter alia) on the local pH level and/or temperature at the drug depot and is released from the drug depot to the circulatory system.

The long action of the insulin detemir results from the addition of fatty acid side chains to native insulin, which stabilizes its self-association into hexamers and permits reversible insulin-albumin binding. When insulin detemir is injected to the subcutaneous tissue it aggregates into hexamers at the drug depot. The insulin detemir slowly dissociates into dimers and monomers, which are then absorbed in the bloodstream. Once in the circulation, insulin detemir may be 98% albumin bound, which also contributes to its protracted action.

One of the main drawbacks of exogenous insulin therapy compared to normal physiology is its increased variability in terms of the pharmacokinetic and pharmacodynamic profile during the lifetime the long acting insulin is active in the patient, leading to an unpredictable effect of the drug. Additionally, variability may be caused by fluctuations in basal insulin pharmacokinetic and pharmacodynamic profiles, which can be inherent to the absorption process. The basal insulin can take a few hours to reach an insulin plateau, which following thereof, the insulin plateau can decrease towards the end of the lifetime of the basal insulin before receiving a new drug injection.

Moreover, upon injecting the long acting insulin repeatedly, such as day after day, there is an accumulation of the drug and it takes about 2-4 days to reach a stable insulin concentration. Hence any interference in the long acting insulin absorption at a given day, such as due to illness or failing to inject the long acting-insulin at a given day, may result in fluctuation of basal insulin concentration for several days afterwards.

The variability in the pharmacokinetic and pharmacodynamic profile may result in any one of the following: increased risk of hypoglycaemia or hyperglycaemia; increased weight gain associated with defensive eating to prevent hypoglycemia; changes in appetite due to fluctuations in glucose or insulin levels; reduced patient confidence in their treatment due to variability in the glucose levels; increased risk of development and/or progression of diabetes complications; and increased risk of mortality.

### SUMMARY OF SOME OF THE EMBODIMENTS

Managing illnesses, particularly chronic illnesses, require monitoring at all times to prevent the onset of a medical risk associated with the deviation of a monitored analyte level from a normal, healthy level.

The normal, healthy analyte level may be measured by a discrete, predetermined level or by a predetermined range comprising a lower threshold and an upper threshold. Upon deviation from the predetermined level or range it is desired to release the long (or short) acting drug into the circulatory system to normalize the analyte level to return to the healthy level or range.

According to an embodiment of the present application, there is provided a method, system and apparatus for regulating the release rate of the long (or short) acting drug in accordance with a detected analyte level so as to ensure the analyte level will remain at or within the predetermined level or range. The drug (long or short acting) release rate is controlled by a treatment apparatus.

In some embodiments, the treatment apparatus comprises a cooling element for cooling a drug delivery site, thereby halting or decreasing the release rate of the drug from the drug depot into the circulatory system. In some embodiments, the treatment apparatus comprises a heating element in addition to the cooling element for heating the drug delivery site, thereby commencing or increasing the release rate of the drug from the drug depot into the circulatory system.

In some embodiments, the treatment applied by the treatment device is governed by methods comprising protocols and algorithms developed for activating the treatment to maintain the analyte level within the predetermined level or range and to prevent an onset of a medical risk.

Furthermore, during times the patient is unaware, such as while sleeping or for patients that are unable to proactively manage their condition, such as children, precautionary measures must be taken to prevent the onset of a medical risk associated with the deviation of the monitored analyte from a normal, healthy level.

There is thus provided in accordance with an embodiment of the present disclosure a drug delivery control apparatus (e.g. a treatment apparatus) configured to control an amount of drug contained in a drug depot delivered or otherwise perfused or diffused into the circulatory system of a patient comprising a cooling element configured for cooling a treatment area by removing heat from the treatment area. The cooling element may be arranged above or near the treatment area. A heat disposal assembly is in thermal communication with the cooling element and configured for directing the removed heat to a heat zone away from the treatment area. A power source, a controller and a housing may be configured to at least partially house at least the cooling element and the heat disposal assembly.

In some embodiments, the cooling element comprises a thermoelectric cooler having at least a first plate and a second plate. The heat disposal assembly may comprise a thermal conducting adhesive configured to direct heat to the heat zone, and a heatsink in thermal contact with the first plate of the thermoelectric cooler.

In some embodiments, the heat disposal assembly comprises a fan.

In some embodiments, the controller is configured to control the thermoelectric cooler to apply heat or cooling to the treatment area.

In some embodiments, the heat zone is spaced away from the treatment area between 2 to 5 centimetres.

In some embodiments, the housing comprises a thermal conductive case. The housing may include a plurality of at least one of folds and creases.

In some embodiments, the heat disposal assembly comprises a phase change material configured to absorb at least some heat from the treatment area.

In some embodiments, the treatment area comprises a drug delivery site for delivery and storage of a drug to a drug depot comprising an area within a subcutaneous tissue layer proximate the drug delivery site, and the apparatus is configured to heat or cool the treatment area, such that a change of the local tissue and local circulatory system properties affecting the drug contained within the drug depot, is established.

In some embodiments, the apparatus further comprises at least one sensor configured to determine at least one analyte level, wherein when the analyte level deviates from a predetermined range, the controller activates a treatment protocol to effect heating or cooling of the treatment area.

In some embodiments, the cooling element comprises a thermally conductive plate, the thermally conductive plate is arranged above or adjacent the treatment area. The heat disposal assembly comprises a phase change material, and the apparatus further comprises a thermal switch provided between the thermally conductive plate and the phase change material.

In some embodiments, the thermal switch comprises a mechanical pin, wherein the mechanical pin is configured to establish thermal contact between the thermally conductive plate and the phase change material. When the mechanical pin establishes thermal contact, the thermally conductive plate cools down.

In some embodiments, the phase change material is contained within thermal insulation. The thermal insulation may comprise a vacuum.

In some embodiments, the thermal switch may comprise an enclosure arranged between the phase change material and the thermally conductive plate. The enclosure is at least partially filled with a fluid to selectively limit thermal contact between the phase change material and the thermally conductive plate. The controller may be configured to increase the temperature of the thermally conductive plate.

In some embodiments, the apparatus comprises a first unit comprising at least the cooling element. The first unit may be arranged above or near the treatment area. A second unit comprises at least the power source and the controller. The second unit may be arranged away from the treatment area. The first unit and the second unit may be thermally connected via at least one conduit.

There is thus provided in accordance with an embodiment of the present disclosure a drug delivery method configured to deliver or otherwise perfuse or diffuse a drug from a drug depot. The drug depot comprises a drug stored within a subcutaneous area of tissue beneath a treatment area. The method comprises providing an apparatus configured to heat or cool the treatment area, the apparatus including a controller in communication with least one sensor configured to sense the concentration level of at least one analyte; determining, based on data obtained from the at least one sensor over a time period, a trend of the concentration level of the at least one analyte based on the obtained data over the time period; determining, based on the trend, a protocol to apply treatment to the treatment area, the treatment protocol including at least one of an operational mode, a temperature, and a duration of treatment; initiating treatment to apply the treatment to the treatment area according to the protocol.

In some embodiments, at least one analyte comprises blood glucose and the sensor comprises a blood glucose sensor, and wherein the trend comprises the concentration of blood glucose level over the time period. The trend may be calculated by adding a current blood glucose concentration level measurement to a change in blood glucose concentration level over time.

In some embodiments, when the trend is below a predetermined threshold, the treatment protocol comprises cooling the treatment area, and when the trend is above a predetermined threshold, the treatment protocol comprises heating the treatment area.

In some embodiments, when cooling is applied to the treatment area, the method further comprises stopping cooling once the trend is above a second predetermined threshold. When heating is applied to the treatment area, the method further comprises stopping heating once the trend is below a second predetermined threshold.

In some embodiments, at least one sensor comprises a blood glucose sensor, and wherein the trend is an average blood glucose concentration level over a period of time.

In some embodiments, at least one sensor comprises a blood glucose sensor.

In some embodiments, the trend is a rate of change of blood glucose concentration level.

In some embodiments, once the treatment protocol is activated, the method further comprises determining, based on data obtained from the at least one sensor, a second trend based on data obtained from the at least one sensor after initiation of the treatment protocol, comparing the trend to the second trend, determining, based on the comparison of the trend with the second trend, a secondary treatment protocol including at least one of a second operational mode, a second temperature, and a second duration of treatment; and applying the secondary treatment protocol to the treatment area.

The determination of at least one of the trend and the treatment protocol may be based in part on data specific to a patient receiving treatment. The data specific to the patient may comprise the medical history of the patient.

The temperature corresponding to the treatment protocol and the second temperature corresponding to the secondary treatment protocol are within an effective temperature range.

The effective temperature range is between about 30° C. and about 42° C.

The effective temperature range comprises at least one optimal temperature effective for diffusion or perfusion of the drug into the patient's circulatory system.

In some embodiments, a treatment protocol is determined based on at least one of: the drug, patient data, statistics, data inputted into the controller, data received from at least one biosensor, and historical data received from the at least one biosensor.

In some embodiments, the treatment protocol includes at least one of heating the treatment area and cooling the treatment area.

In some embodiments, the treatment protocol is determined based on patient-specific data. The patient-specific data may comprise medical history. The patient-specific data may comprise patterns, trends, reactions, and characteristics of the user. The patient-specific data may be detected by at least one biosensor and stored in a memory by the controller.

In some embodiments, at least one biosensor determines the analyte level at predetermined intervals, and wherein the controller determines an analyte level pattern. The analyte level pattern corresponds to past analyte level measurements at the predetermined intervals, and wherein the controller is further configured to determine a trend of anticipated analyte levels.

In some embodiments, when the controller determines that the trend of anticipated analyte levels is outside of the predetermined analyte level range, the controller activates a treatment protocol. The treatment protocol is different depending on the severity of the trend.

In some embodiments, the apparatus may include at least one biosensor configured to determine an activity level of a patient, and wherein the treatment protocol varies depending on the determined activity level.

The controller determines, based on the trend of anticipated analyte levels, an anticipated time when the anticipated analyte levels will be outside an acceptable range of analyte levels.

The controller determines a treatment protocol based on the anticipated time that the anticipated analyte levels will be outside the acceptable range, and wherein the controller activates the treatment protocol.

In some embodiments, heat flows from the second plate of the thermoelectric cooler to the first plate of the thermoelectric cooler in response to an electric current. The flow of heat from the second plate to the first plate cools the treatment area. When direction of the electric current is switched, heat flows from the first plate of the thermoelectric cooler to the second plate of the thermoelectric cooler. The flow of heat from the first plate to the second plate may heat the treatment area. The electric current generated by a temperature difference between the first plate and the second plate may be used to charge a battery associated with the power source.

### BRIEF DESCRIPTION OF THE DRAWINGS

The principles and operations of the systems, apparatuses and methods according to some embodiments of the present disclosure may be better understood with reference to the drawings, and the following description. The drawings are given for illustrative purposes only and are not meant to be limiting.

FIGS. 1A-1C are an illustration of an exemplary system for controlling the absorption of a drug, according to some embodiments of the present disclosure;

FIGS. 2A and 2B are illustrations of an exemplary system for controlling the absorption of a drug (FIG. 2A) and a cross section thereof (FIG. 2B), according to some embodiments of the present disclosure;

FIGS. 3A and 3B are illustrations of an exemplary system for controlling the absorption of a drug (3A) and a cross section thereof (FIG. 3B), according to some embodiments of the present disclosure;

FIGS. 4A and 4B are illustrations of an exemplary system for controlling the absorption of a drug (4A) and a cross section thereof (FIG. 4B), according to some embodiments of the present disclosure;

FIGS. 5A and 5B are illustrations of an exemplary system for controlling the absorption of a drug (5A) and a cross section thereof (FIG. 5B), according to some embodiments of the present disclosure;

FIGS. 6A and 6B are illustrations of an exemplary system for controlling the absorption of a drug (6A) and a cross section thereof (FIG. 6B), according to some embodiments of the present disclosure;

FIGS. 7A and 7B are illustrations of an exemplary system for controlling the absorption of a drug (7A) and a cross section thereof (FIG. 7B), according to some embodiments of the present disclosure;

FIGS. 8A and 8B are illustrations of an exemplary system for controlling the absorption of a drug (8A) and a cross section thereof (FIG. 8B), according to some embodiments of the present disclosure;

FIGS. 9A and 9B are illustrations of an exemplary system for controlling the absorption of a drug (9A) and a cross section thereof (FIG. 9B), according to some embodiments of the present disclosure;

FIGS. 10A and 10B are illustrations of an exemplary system for controlling the absorption of a drug (10A) and a cross section thereof (FIG. 10B), according to some embodiments of the present disclosure;

FIGS. 11A and 11B are illustrations of an exemplary system for controlling the absorption of a drug (11A) and a cross section thereof (FIG. 11B), according to some embodiments of the present disclosure;

FIGS. 12A and 12B are illustrations of an exemplary system for controlling the absorption of a drug (12A) and a cross section thereof (FIG. 12B), according to some embodiments of the present disclosure;

FIG. 13 is a cross sectional illustration of an exemplary system for controlling the absorption of a drug, according to some embodiments of the present disclosure;

FIG. 14 is a cross sectional illustration of an exemplary system for controlling the absorption of a drug, according to some embodiments of the present disclosure;

FIG. 15 is a cross sectional illustration of an exemplary system for controlling the absorption of a drug, according to some embodiments of the present disclosure;

FIG. 16 is a flowchart of an exemplary method for controlling the absorption of a drug, according to some embodiments of the present disclosure;

FIG. 17 is a graph showing changes in blood glucose levels over time while applying treatment according to a protocol based on an analyte level trend, according to some embodiments of the present disclosure;

FIG. 18 is a flowchart of an exemplary method for controlling the absorption of a drug, according to some embodiments of the present disclosure;

FIG. 19 is a graph showing results of a study of the effect of applying a treatment for controlling the absorption of a drug on an analyte level;

FIG. 20 is a graph showing results of a study of the effect of applying a treatment for controlling the absorption of a drug on an analyte level;

FIG. 21 is an illustration of an exemplary system for regulating the absorption of a drug, according to some embodiments of the present disclosure;

FIG. 22 is an illustration of an exemplary system for controlling the absorption of a drug, according to some embodiments of the present disclosure; and

FIG. 23 is an illustration of an exemplary system for controlling the absorption of a drug, according to some embodiments of the present disclosure.

### DETAILED DESCRIPTION OF SOME OF THE EMBODIMENTS

According to some embodiments of the present disclosure, there is provided a system for controlling the absorption of a drug (long acting or short acting) into the circulatory system via capillaries to the cardiovascular system, and/or the lymphatic system. The absorption of the drug may be controlled by controlling the release rate of the drug from the drug depot into the circulatory system, which comprises the cardiovascular system and the lymphatic system. The system may include a treatment device comprising a treatment element configured for increasing or decreasing the drug absorption.

In some embodiments, the treatment device may include a thermal stimulator, such as a cooling element configured to cool a delivery site of the drug, thereby cooling the drug depot and thus decreasing the absorption of the drug.

It was found, and will be described in reference to FIGS. 17, 19 and 20, that cooling the drug delivery site can decrease the absorption of a long acting insulin-containing drug, resulting in an increase in the blood glucose level, thus preventing or minimizing the risk of hypoglycemia. Heating the drug delivery site can increase the absorption of a long acting insulin-containing drug, resulting in a decrease in the blood glucose level, thus preventing or minimizing the risk of hyperglycemia.

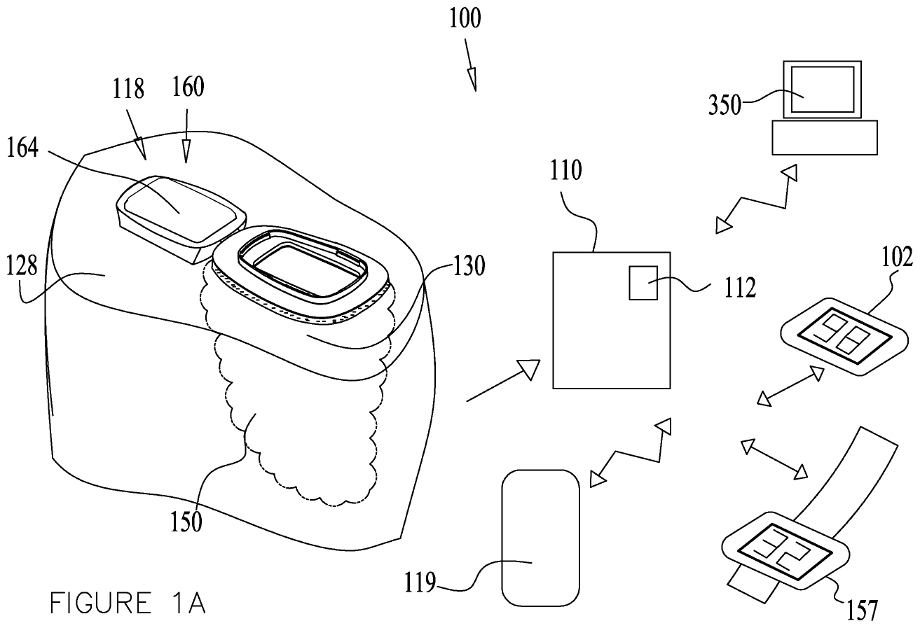

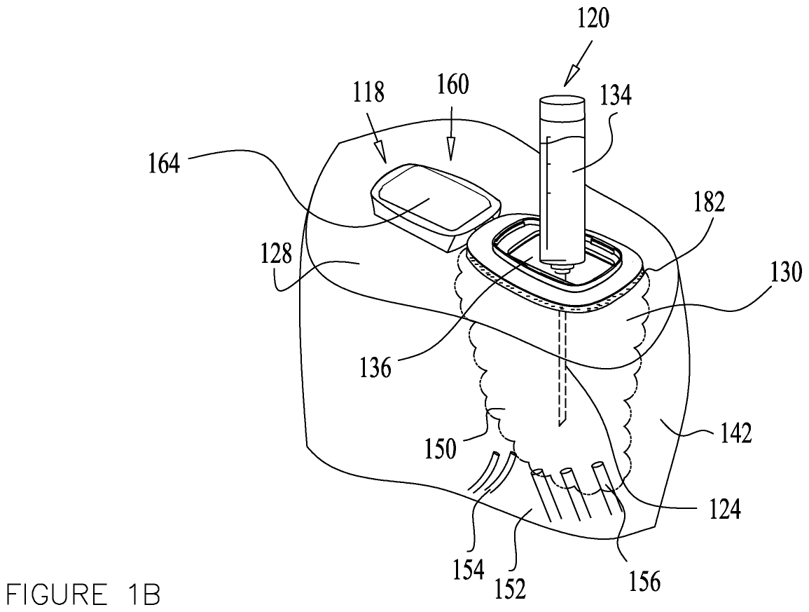

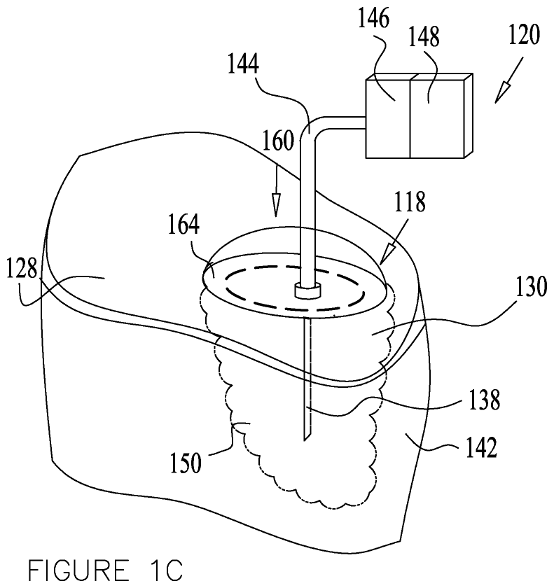

As seen in FIG. 1A, a system 100 for controlling the absorption of a drug comprises a biosensor 102 configured to be engaged with a patient (e.g. a user). The biosensor 102 may be conditioned to monitor a certain bodily analyte, with or without the patient intervention, such as continuously or at predetermined intervals. The detected analyte level is transmitted to a system controller 110 by any suitable means. The analyte level may comprise the concentration of the analyte in the blood or another body tissue.

Upon detection of a current deviation or of a potential future deviation of the detected analyte level from the predetermined analyte level or range, the system controller 110 may initiate application of a treatment by a treatment element 118 (typically part of a treatment apparatus 160) which may be activated by the user or automatically, such as via the system controller 110, to decrease (or increase) the absorption of the drug to prevent or correct the deviation from the predetermined analyte level or range.

In some embodiments, the system controller 110 may initiate an alert directed to the patient or any other caregiver, through a user interface 119, which can include a visual display and/or audio means generating a sound, so that the user can be properly alerted and take measures to prevent deviation from the predetermined analyte level by normalizing the analyte level. The alert may be generated by any suitable means, such as by an audio or visual signal generated by the treatment apparatus 160 or any other device.

The user interface 119 may comprise a device with a processor such as a computer having a display device (e.g., a LCD (liquid crystal display) monitor and the like) for displaying information to the user and a keyboard and/or a pointing device e.g., a mouse, trackball or a touchscreen, by which the user may provide input to the computer. For example, user interface 119 may comprise a dispensing unit, remote control, PC, laptop, smartphone, media player or personal data assistant (“PDA”). Other kinds of devices may be used to provide for interaction with the user, as well.

Exemplary biosensors 102 may include glucose monitors, continuous glucose monitors, heart rate monitors, ECG monitors, pulse oximeters, blood pressure monitors, respiration rate monitors, EEG monitors, etc.

Exemplary analytes may include blood glucose, blood pressure, heart rate, lactate, alcohol, triglycerides, cholesterol, HDL, glycerol, etc.

In a non limiting example, the analyte is the blood glucose and the drug is an insulin-containing drug. Deviations from a predetermined healthy blood glucose range may occur due to a multiplicity of causes, such as nutrition and body activity, for example. A high blood glucose level may cause hyperglycemia while a low blood glucose level may cause hypoglycemia. The predetermined blood glucose level or range may be based on known clinical data. For example the lower threshold may be about 70 or about 75 mg/dL indicating hypoglycemia, and the upper threshold may be about 160 or 180 mg/dL indicating hyperglycemia. In some embodiments, to maintain a healthy blood glucose level the predetermined acceptable range may be about 80 to about 140 mg/dL. In some embodiments, to maintain a healthy blood glucose level, the predetermined acceptable range may be about 80 to about 120 mg/dL.

The patient may receive drug therapy (e.g. a long acting drug or a short acting drug) for normalizing the analyte level or range in the body. The drug may be administrated in any suitable manner by a drug delivery device 120.

According to some embodiments, the drug delivery device 120 comprises a syringe or an injection pen, as seen in FIG. 1B, and the drug is administrated by a needle 124 piercing the skin tissue 128 at a drug delivery site 130.

The drug delivery site 130 may be referred to as the “treatment area” or the “drug injection site” or “injection site”.

In some embodiments, the drug may be administrated by injection where the drug flows from a drug reservoir 134 through the needle 124 into a subcutaneous tissue layer 142. In FIG. 1B the drug delivery device 120 is shown injecting the drug through an injection window 136 formed in a treatment apparatus 160, shown at an open state.

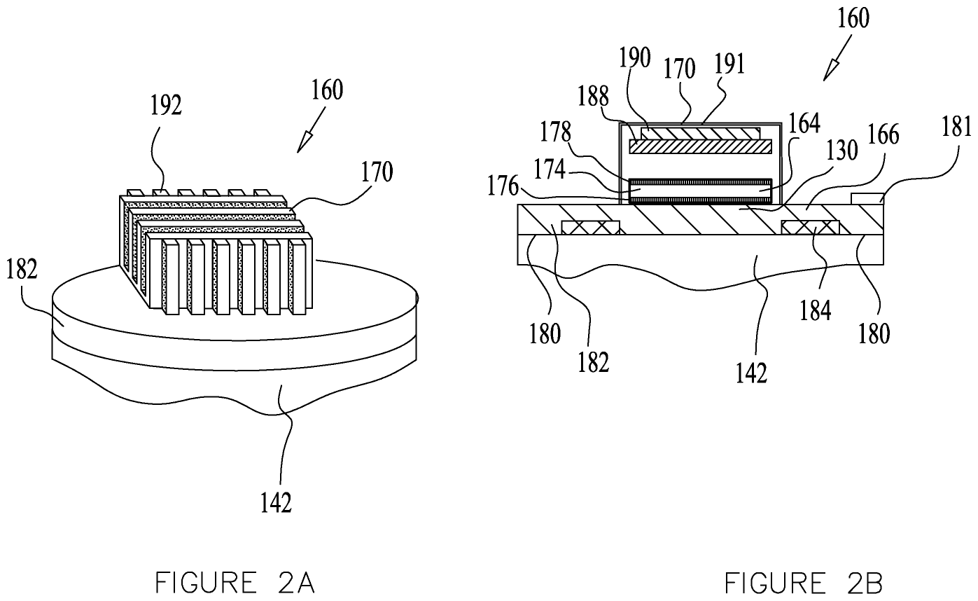

FIGS. 2A-15 show the treatment apparatus 160 in a closed state overlaying the injection window 136. It is appreciated that the treatment apparatus 160 may be formed without an injection window 136 wherein the treatment apparatus 160 may be placed on the drug delivery site 130 after drug delivery or prior to drug delivery.

In other embodiments, the drug delivery device 120 comprises an infusion set, as seen in FIG. 1C, and the drug may be administrated by infusion where a cannula 138 can be inserted at the drug delivery site 130. The drug may be infused to the subcutaneous tissue layer 142 via a catheter 144.

In some embodiments, the catheter 144 may be connected at a second end thereof to a drug reservoir 146. The infusion set may comprise an infusion pump 148, provided for control of the drug delivery from the drug reservoir 146. In some embodiments, the infusion pump 148 may be obviated.

The treatment element 118 may be placed at any suitable location. As seen in FIG. 1C, the treatment element 118 may be configured in the infusion set and may be connected to the catheter 144. In some embodiments, the treatment element 118 may be disconnected from the catheter 144. In some embodiments, the treatment element 118 may be placed on the catheter 144 or in proximity thereto.

In both injection and infusion administration, the drug reaches a drug depot 150. The drug flows thereafter into the circulatory system 152, via capillaries 154 of the cardiovascular system and/or via the lymphatic system 156 (FIG. 1B).

In some embodiments, the drug depot 150 may comprise an area of tissue surrounding the needle 124 or cannula 138. This tissue may comprise subcutaneous tissue 142.

In some embodiments, the system 100 may comprise a single or a plurality of additional sensors 157 for detecting signals utilized by the controller 110 in determining the treatment and the treatment parameters, e.g. temperature, duration, time of activating the treatment etc.

In some embodiments, the sensors 157 may be configured for detecting a signal indicating a bodily-function of the patient. In some embodiments, the bodily function sensor 157 may comprise an activity level sensor configured to detect a bodily function other than the analyte level, which may or may not be related to the analyte level. In a non-limiting example, the bodily-function sensor 157 may comprise an activity level sensor configured to detect an activity level of the patient indicting the degree of energy expenditure exercised by the patient. The activity level sensor may comprise a single or plurality of sensors for detecting any one of: the state of consciousness of a patient i.e. is the patient awake or asleep and/or the degree of energy expenditure exercised by the patient, since at times the energy expenditure degree can affect the analyte level.

The bodily-function sensor 157 may comprise further sensors detecting glucose-effecting activities, such as meals and nutrition, for example.

In some embodiments, the bodily-function sensor 157 may comprise a temperature sensor configured to detect the temperature of the skin surface 128 at the drug delivery site 130.

A non-limiting example of bodily function sensor 157 is the activity level sensor, which may comprise a pedometer, a heart rate meter or any other suitable device. Activity level sensor 157 may be embedded in the treatment element 118, in the user interface 119 or in a separate device, as shown in FIG. 1A.

In some embodiments, nutrition related information, such as the time a meal was consumed, the content (e.g. carbohydrate, protein and fat content) and/or the meal duration, may be provided to the system 100. The nutrition information may affect the analyte level, for example, consumption of carbohydrates may raise the blood glucose level. The nutrition information may be automatically detected by a meal detector, such as disclosed in Applicants' PCT Publication WO2011/016028, the disclosure of which is expressly incorporated herein by reference in its entirety. In some embodiments, the user may enter the information via the user interface 119. In some embodiments the user interface 119 or any other device may be programmed to prompt the user to enter the nutrition information, such as by displaying a reminder on the user interface screen or generating an alert, for example.

In some embodiments, data pertaining to drug related information may be provided by additional sensors 157 and/or by the user who may enter the information via the user interface 119. The drug related information may include time passed from previous long acting drug delivery and/or its dose and/or the drug composition, data pertaining to time past from previous short acting drug delivery, or bolus injection and/or its dose and/or the drug composition.

In some embodiments, the controller 110 may comprise a timing functionality including a timer for determining the time passed since a previous event (e.g. injection, meal, activity) or a timer for applying the cooling or heating for the predetermined duration.

In some embodiments, the controller 110 may comprise a memory module 112 for data storage and retrieval.

The system controller 110 may be configured to receive data and/or signals from any one of the system components and devices, such as the biosensor 102, additional sensors 157 and/or the user interface 119. Based on the received data the system controller 110 may be configured to anticipate a future change in the analyte level, such as a potential deviation of the analyte level from the predetermined analyte level or range.

In some embodiments, upon detection of the deviation from the predetermined analyte level or anticipation of a future deviation from the predetermined analyte level, the system controller 110 may be configured activate the treatment element 118 to apply a treatment for affecting the release rate of the drug, thereby preventing or correcting the deviation of the analyte level from the predetermined level or range. The drug release rate may be increased or decreased according to the type of drug and its effect on the analyte level.

In some embodiments, the drug release rate may be decreased or halted by cooling. The drug release rate may be increased or commenced by heating.

Cooling may comprise achieving a temperature below the body temperature at the drug injection site 130 for cooling the drug depot 150. Heating may comprise achieving a temperature above the body temperature at the drug injection site 130 for heating the drug depot 150.

In some embodiments, cooling may comprise cooling to a temperature below a predetermined temperature range or threshold. It was discovered that the blood perfusion and the local lymphatic system perfusion mainly occurs within a “drug release effective temperature range” e.g. 30° C.-42° C. Namely, the long acting drug is mainly released from the drug depot at this “effective temperature range.” Accordingly, cooling may comprise achieving a temperature below the effective temperature range at the injection site 130 and heating may comprise achieving a temperature within or near the upper threshold of the effective temperature range or above the effective temperature range.

There are some types of chemically transitioning drugs which proceed through a cascade of chemical states from drug delivery until absorption into the circulatory system 152. The long acting insulin is such a drug, as described herein. It was discovered for these drugs that the decrease of the chemical state transition process mainly occurs within a physiological range of the “effective temperature range.” In a non limiting example the effective temperature range was found to be at 20° C.-42° C. or at subranges thereof.

The treatment element 118 can be configured to increase or decrease the delivery rate of the drug into the circulatory system 152 by application of a treatment, via the surface of the skin 128.

The treatment element 118 can be placed at any suitable location. For example, the treatment element 118 can be placed on the skin surface 128 or in proximity thereto. In some embodiments, the treatment element 118 can be arranged in proximity to the drug delivery site 130. In some embodiments, the treatment element 118 can be placed away from the drug delivery site 130.

In some embodiments the treatment element 118 may be realized by a treatment apparatus 160. In some embodiments, the treatment apparatus 160 comprising the treatment element 118 may comprise a cooling element 164 configured to cool the delivery site 130 for decreasing the delivery rate of the drug from the drug depot 150 into the circulatory system 152.

In some embodiments, the cooling element 164 may comprise elements for removing heat from the injection site 130, thereby cooling the drug depot 150. Removal of heat may be formed in any suitable manner, some exemplary embodiments are described in reference to FIGS. 2A-15.

Cooling by the cooling element 164 may be applied to the skin surface 128 before, during and/or after the delivery (e.g. injection or infusion) of the drug is administrated. The treatment apparatus 160 may remain on the skin surface 128 for a selected time period.

In the embodiment of FIG. 1B, further injections of the drug may be administrated at the drug delivery site 130 through injection window 136.

As seen in FIGS. 2A-15, the cooling element 164 may be enclosed within a housing 170. The cooling element 164 may comprise any suitable heat removal apparatus, such as a thermoelectric cooler (TEC) 174. The thermoelectric cooler 174 utilizes the Peltier effect, which in response to an electric current, heat flows from a bottom plate 176, here shown to be arranged in proximity to the injection site 130 to an upper plate 178, here shown to be arranged distally to the injection site 130, thereby removing heat from the injection site 130.

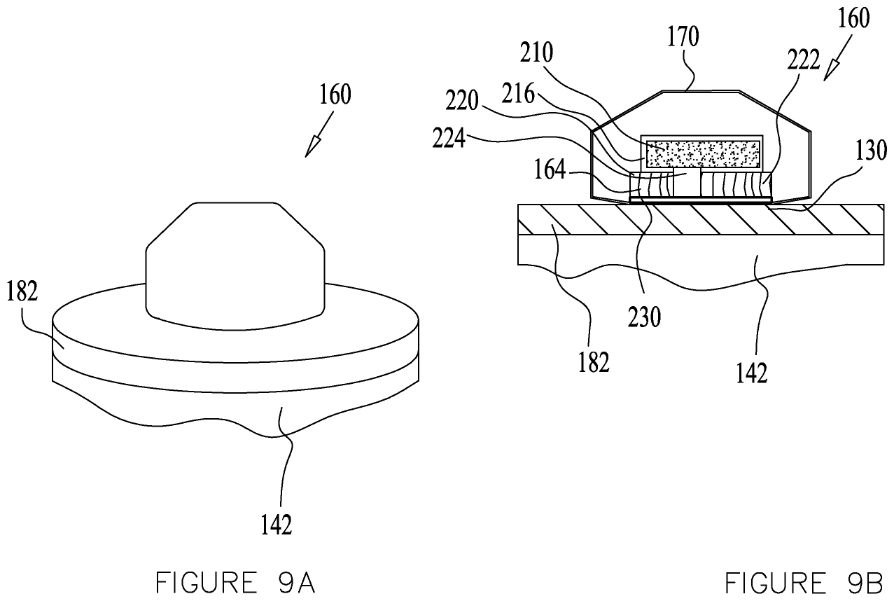

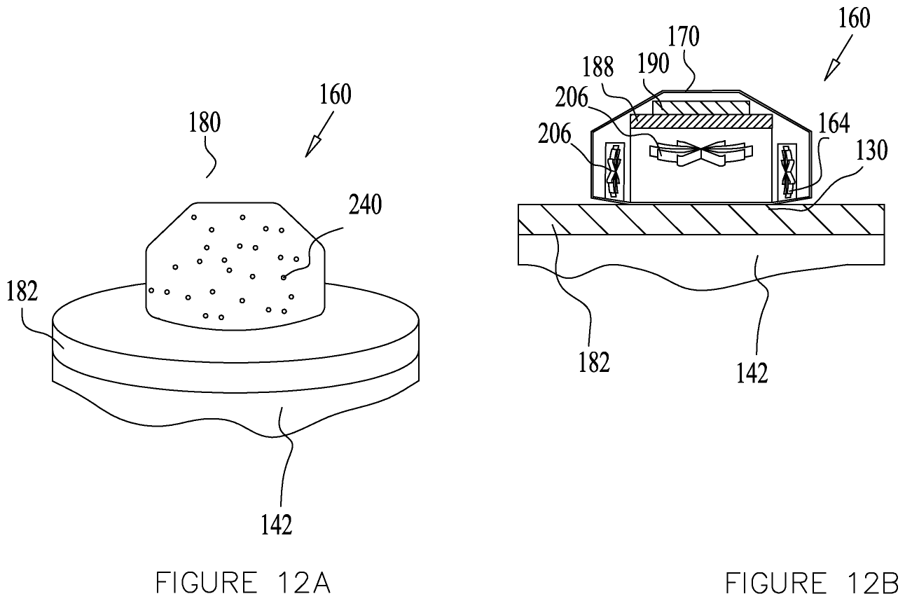

Further non-limiting examples for the cooling element 164 are a cooling refrigerator, a heat pump for heat removal from the injection site, a cooling material (e.g. a gel, liquid or solid), a cryogenic material, a fan (FIGS. 12A and 12B), a phase change material (PCM) (FIGS. 9A and 9B), a resistor, ducts flowing with a cooling fluid, such as water, acetone, nitrogen, methanol, ammonia or sodium, for example and/or any combination thereof. In some embodiments, the cooling element 164 may comprise a cooling unit for applying direct cold to the injection site 130. The cooling unit may comprise ice, a chemical cooling agent or any other suitable means.

The treatment apparatus 160 may comprise a heat disposal assembly 166 in thermal communication or contact with the cooling element 164 for directing the removed heat, removed by the cooling element 164, away from the drug delivery site 130.

In some embodiments the treatment apparatus 160 is placed on the injection site 130 under clothing. Removed heat from the injection site 130 may be trapped by the clothing and inadvertently reabsorbed by the injection site 130 or in proximity thereto and interfere during the cooling mode. Furthermore the removed heat may also affect temperature sensitive drugs, e.g. insulin, which have an efficacious temperature limit and are degraded by overheating. Therefore directing the removed heat away from the injection site 130 is provided by the heat disposal assembly 166.

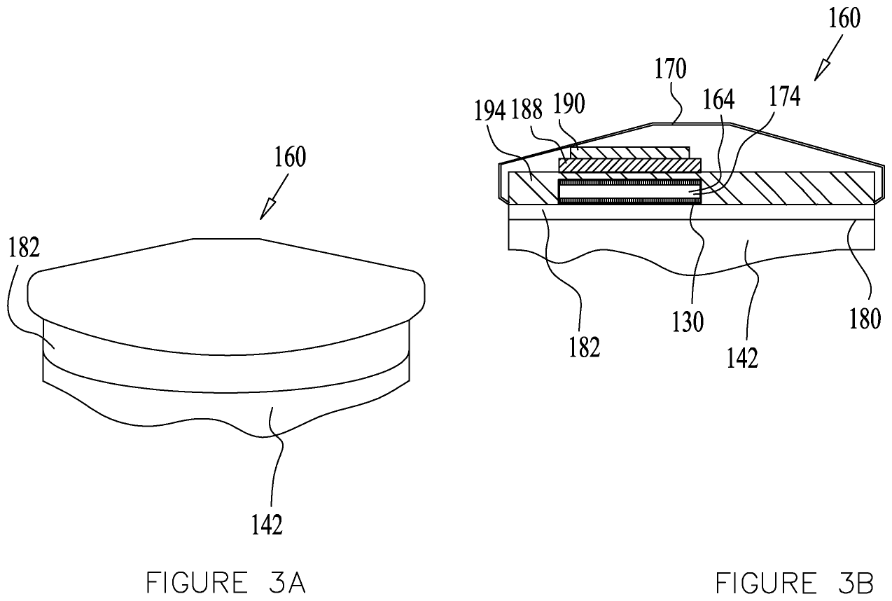

In some embodiments (e.g. FIGS. 2A-3B), to minimize heat absorption at the injection site 130, when cooling is desired, the heat disposal assembly 166 may include directing the removed heat to a “heat zone” 180 positioned sufficiently distally from the injection site 130, where heat is absorbed in the body tissue yet does not affect the temperature at the injection site 130. In some embodiments, the sufficient distance may be about 2 or more centimeters away from the injection site. In some embodiments this distance may be about 3 or more centimeters away from the injection site. In some embodiments this distance may be about 4 or more centimeters away from the injection site. In some embodiments this distance may be about 5 or more centimeters away from the injection site. The heat zone 180 may surround or may be symmetrically arranged about the injection site 130, such as seen in FIG. 2B. In other embodiments, such as shown in FIG. 3B, the heat zone 180 may be arranges at one side of the injection site 130.

In some embodiments, to enable efficient heat absorption by the body at the distal location, i.e. the heat zone 180, the skin temperature at the heat zone may be kept at temperatures most efficient for heat absorption, such as about 37° C. to about 42° C., by using an additional thermoelectric cooler or other cooling element 181 designed to heat the skin to an efficient heat absorption temperature (e.g. about 40° C.) or using a PCM with phase transition temperature matching the efficient heat absorption temperature, for example.

In some embodiments, the heat zone 180 may include the ambient environment external to the treatment device. As seen in FIG. 2B the heat disposal assembly 166 may direct the removed heat to the heat zone 180 in the ambient environment out of the treatment apparatus 160.

The removed heat may be directed to the heat zone 180 in any suitable manner. In the exemplary embodiment of FIGS. 2A and 2B, an adhesive 182 or any other attachment means connecting the treatment apparatus 160 to the skin 128, may be formed at least partially of a thermally conducting material configured to conduct the removed heat from the injection site 130 to the heat zones 180. The adhesive 182 may further be formed with a thermally isolating portion 184 arranged intermediate the injection site 130 and the heat zone 180, thereby preventing the removed heat from being reabsorbed into the tissue in proximity to the injection site 130.

Power supply to the treatment apparatus 160 may be provided in any suitable means, such as a rechargeable or a disposable battery 188 positioned in any suitable location in the treatment apparatus 160 or in conjunction thereto. The treatment apparatus 160 may comprise a controller and electrical contacts 190 for controlling and activating the treatment element 118 (e.g. the cooling assembly 164) and in some embodiments for controlling communication with other devices, as described in reference to FIG. 1A. The controller and electrical contacts 190 may be arranged within the treatment apparatus 160 or in conjunction thereto.

It is noted that in some embodiments the system controller 110 (FIG. 1A) may be external to the treatment apparatus 160 and may embed the apparatus controller 190 therein. In some embodiments, the system controller 110 may be external to the treatment apparatus 160 and the controller 190 may be embedded in the treatment apparatus 160. In some embodiments, the system controller 110 may be embedded in the treatment apparatus 160 along with the controller 190. In some embodiments, the system controller 110 may comprise the apparatus controller 190.

In the embodiments of FIGS. 2A-15 the battery 188 and the controller and electronics 190 may be placed within the treatment apparatus 160 at a location unaffected by the removed heat, such as near the roof 191 of housing 170.

In some embodiments, the housing 170 of any of the treatment devices 160 described herein may be structured for enhanced heat removal therefrom. The housing 170 may be formed as a thermal conductive case to allow the heat to dissipate therefrom by radiation and convection, such as with an increased surface area, e.g. with folds and creases. Such an exemplary structure is shown in FIG. 2A wherein the housing 170 is formed with fins 192 in a radiator-like structure. Alternatively and/or additionally, the housing 170 may be painted with paint comprising a high emissivity coefficient or the housing 170 may be covered by a material with a high emissivity coefficient.

Turning to FIGS. 3A and 3B, it is seen that the removed heat may be directed by heat disposal assembly 166 to the heat zone 180 by a thermal plate 194. A portion of the thermal plate 194 may be arranged above the thermoelectric cooler 174 and may extend over to the heat zone 180 or may be placed at any other suitable location.

The adhesive 182 may be formed of any suitable material possibly with non-insulating properties to allow the cooling of the injection site and direction of the removed heat to heat zone 180 by the thermal plate 194. The housing 170 may be formed to enclose the thermal plate 194 and overlie the heat zone 180.

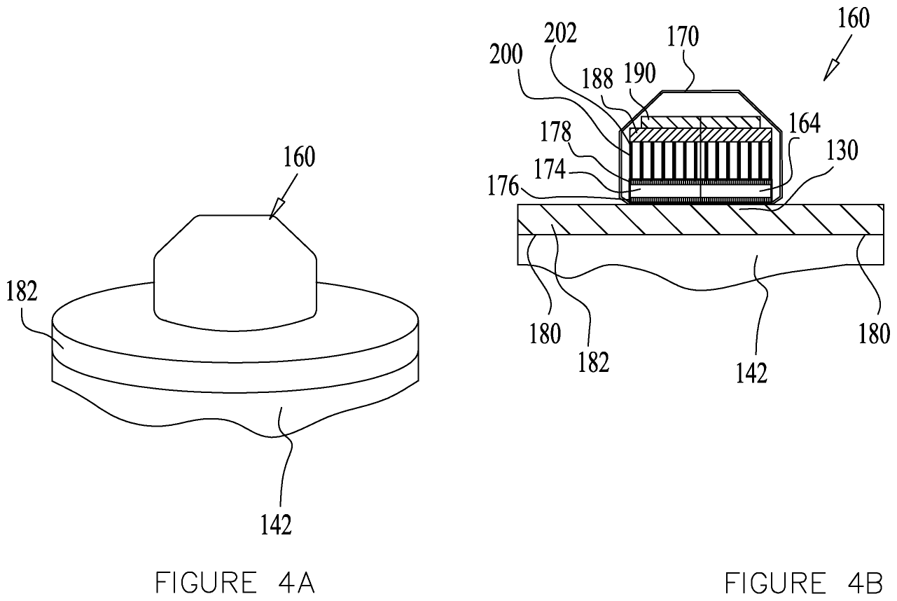

In some embodiments, substantially all the heat is removed to the body tissue. In some embodiments, a portion of the heat is directed to the body tissue and a portion of the heat dissipates into the ambient by convection, radiation or by diffusion. In some embodiments, all the heat is allowed to dissipate into the ambient, such as shown in FIGS. 4A-15.

In FIGS. 4A and 4B the heat disposal assembly 166 may comprise a heatsink 200. The heatsink 200 may be arranged in conjunction and in thermal contact with the thermoelectric cooler 174 for removal of heat therefrom via the heatsink fins 202. In some embodiments the heatsink 200 may be arranged above the thermoelectric cooler 174. In some embodiments, alternatively or additionally to the heatsink 200, there may be provided any suitable heat exchanger transferring the heat from the injection site 130 away from the treatment apparatus 160.

The treatment apparatus 160 may comprise the adhesive 182 which may be formed of a thermally conductive material for transferring some heat from the cooling element 164 into the heat zone 180 away from the injection site 130. Alternatively, the adhesive 182 may be formed partially or fully of a thermally insulating material.

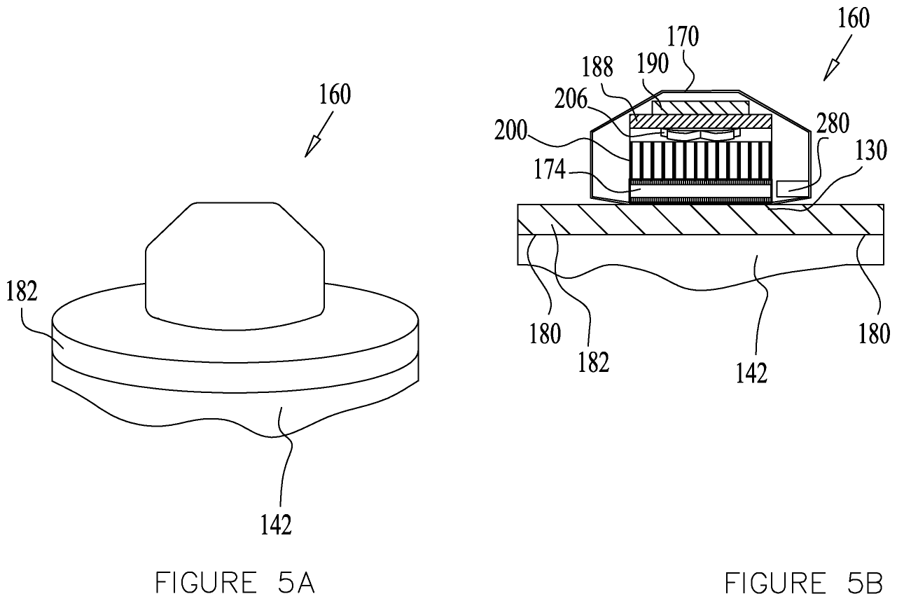

Turning to FIGS. 5A and 5B, it is shown that in addition to heatsink 200 the heat disposal assembly 166 may comprise a fan or blower 206 provided for removal of heat from the treatment apparatus 160. The fan 206 may be arranged above the heatsink 200 and may blow away heat removed by the heatsink 200, as shown in FIG. 5B. The fan 206 may be arranged at any other suitable location, such as at the sides of the treatment apparatus 160 (FIG. 12B).

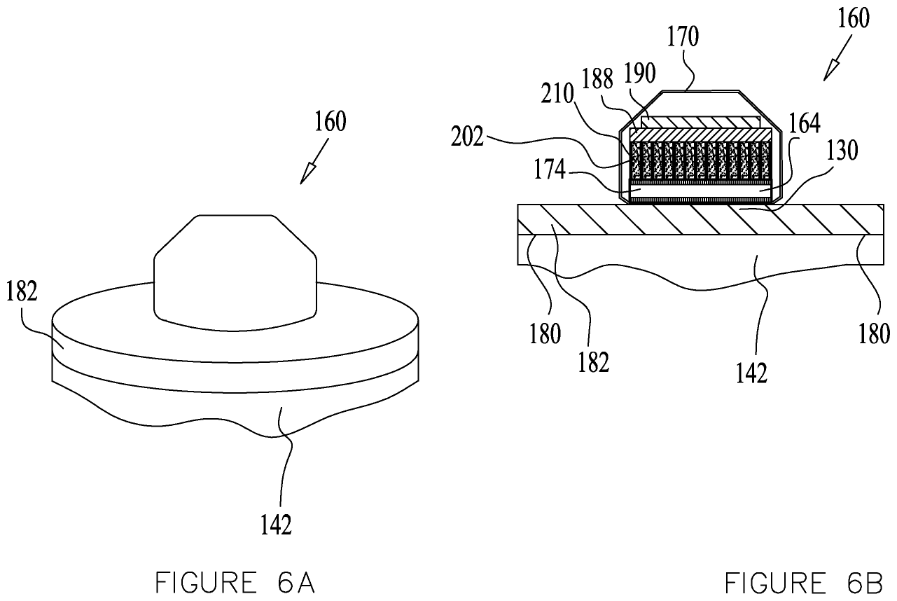

As seen in FIGS. 6A and 6B, in some embodiments the heat disposal assembly 166 may include storing the removed heat from the cooling element 164 in a heat storage apparatus 210. The heat storage apparatus 210 may comprise any heat storage means. The stored heat may be dissipated or may be used to charge the battery 188. In some embodiments, wherein increasing the drug absorption is desired, the stored heat may be utilized to heat the injection site 130 thereby increasing the release rate of the drug from the drug depot 150 into the circulatory system 152.

In some embodiments, the heat storage apparatus 210 may comprise a phase change material (PCM). The PCM 210 is a material with relatively high heat of fusion which, by melting and solidifying at a specific phase transition temperature, is capable of absorbing, storing and releasing relatively large amounts of energy. The PCM 210 may be selected with a phase transition temperature above a predetermined cooling temperature. For example, the selected PCM may have a phase transition temperature of about any one of: about 16° C., about 17° C., about 20° C., about 34° C., about 36° C., about 37° C., about 40° C., or above or below. Thus, upon cooling the injection site 130 below the phase transition temperature, the PCM 210 is in a solid state and the removed heat is absorbed by the PCM 210. When cooling in not required and wherein the temperature of the PCM apparatus 210 rises above the phase transition temperature, the PCM 210 transitions to a liquid state wherein heat is emitted therefrom. In some embodiments, the emitted heat may be used to charge the battery 188 or heat the injection site 130 or may be dissipated to the heat zone 180.

The PCM 210 may be formed of any suitable material, such as paraffin, for example.

The PCM 210 may be arranged at any suitable location within the treatment element 160. In some embodiments, the PCM 210 may be embedded intermediate the heatsink fins 202, as seen in FIG. 6B.

In any one of the embodiments comprising the PCM 210 it is noted that the PCM 210 may comprise more than one type of PCM material, each PCM material characterized by a different phase transition temperature. Thus the cooling during the cooling mode may be performed at different corresponding temperatures. For example, a first PCM may have a phase transition temperature at a relatively high temperature, such as at about 24° C., thus during the cooling mode the injection site 130 may be initially cooled to a first relatively high temperature at about 24° C. A second PCM may have a phase transition temperature at a relatively low temperature, such as at 14° C., thus during the cooling mode the injection site 130 may be further cooled to a second relatively lower temperature at about 14° C.

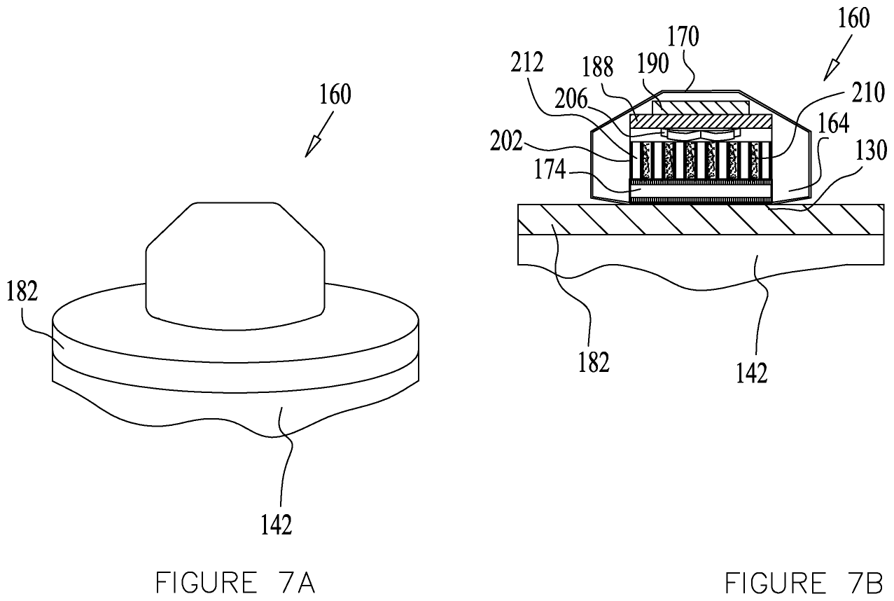

Turning to FIGS. 7A and 7B, it is shown that the PCM 210 is partially arranged within the spaces 212 formed intermediate some of the heatsink fins 202, while some spaces 212 remain vacant (i.e. filled with air) without the PCM 210 embedded therein, to allow the air to blow from the fan 206 through the vacant spaces 212 to remove heat from the treatment apparatus 160.

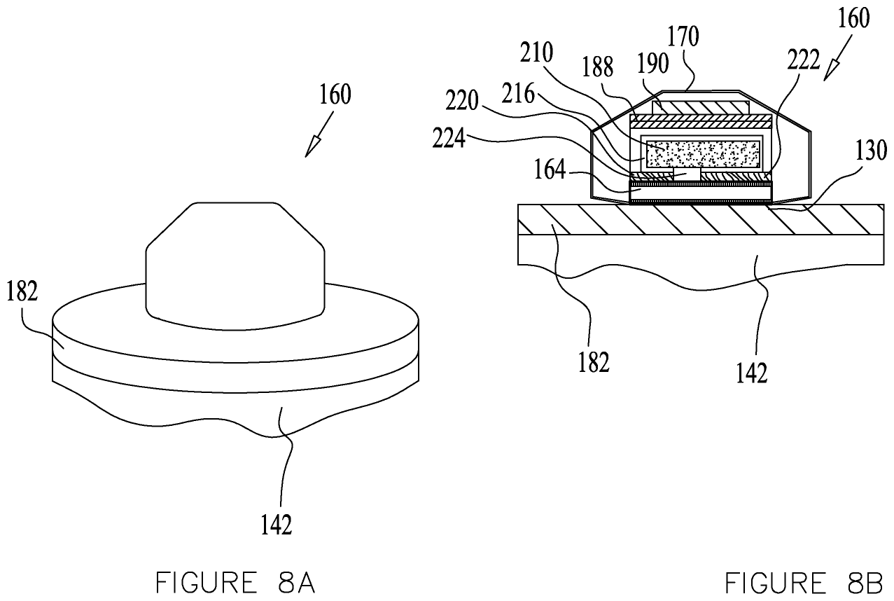

As seen in FIGS. 8A and 8B, in some embodiments, the heatsink 200 may be replaced by the heat storage apparatus 210, such as the PCM 210. The PCM 210 may be placed in a vacuum chamber 216 or any other suitable thermal insulating chamber or layer. The cooling element 164 may comprise the thermoelectric cooler 174 or a metal plate and a resistor or any other cooling mechanism.

In some embodiments, contact between the PCM apparatus 210 and the cooling element 164 may be facilitated by a thermal switch 220 to allow heat to selectively flow from the cooling element 164 to the PCM 210 and to prevent flow of heat when the cooling is not activated.

In some embodiments, the thermal switch 220 may comprise a selectively thermal conduction layer, which may be formed of an additional PCM layer or any other suitable material. The additional PCM layer 222 may be designed to transition from a solid phase to a liquid phase whereupon the PCM storage 210 emits heat, which occurs when the PCM storage 210 transitions from a solid to liquid phase (e.g. about 34° C.). For example, the PCM layer 222 has a phase transition temperature (e.g. about 35° C. or about 37° C. or about 39° C.), which is above the phase transition temperature of the PCM storage 210. Thus when the treatment apparatus 160 applies cooling and the PCM storage 210 is in its solid state, the PCM layer 222 is designed to be in a solid state. This is to allow for good conductivity for flow of the removed heat to the PCM storage 210 from the cooling element 164. When the treatment apparatus 160 halts the cooling and the PCM storage 210 is in its liquid state, the PCM layer 222 is designed to be in a liquid state. This is to provide for poor conductivity for preventing flow of the emitted heat from the PCM storage 210 to the cooling element 164.

In some embodiments, the thermal switch 220 may comprise a contact unit 224 (additionally or alternatively to PCM layer 222). In some embodiments, the contact unit 224 may comprise a movable mechanical pin or arm which may be moved to be in contact with the PCM storage 210, whereupon thermal conductivity is desired. The mechanical pin or arm may be removed from contacting the PCM storage 210 whereupon thermal conductivity is undesired.

In some embodiments, the contact unit 224 may comprise a small enclosure which is filled with a thermally conducting fluid whereupon thermal conductivity is desired, and is evacuated or filled with air, whereupon thermal conductively is undesired.

The movement of the contact unit 224 may be controlled by the controller 110 or 190 via the electrical contacts.

It is noted that the thermal switch 220 may be utilized in any one of the treatment devices 160 described herein.

As seen in FIGS. 9A and 9B, the cooling element 164 may comprise passive elements, such as the PCM 210 and may exclude active cooling elements (e.g. the thermoelectric cooler 174). In some embodiments, the battery 188 and/or the electronics 190 may be obviated and the PCM 210 is selected with a phase transition temperature operative to cool the injection site 130 at the predetermined temperature range. In some embodiments, the battery 188 and/or the electronics 190 may be provided.

A metal plate 230 or any other layer may be arranged intermediate the PCM 210 and the adhesive 182 for applying the cooling (or heating) to the injection site 130. In some embodiments the metal plate 230 may be obviated. The thermal switch 220 may be provided for selective thermal conduction with the metal plate 230 or adhesive 182. The housing 170 may be formed of a thermally insulting material or a low conductivity material since the heat is absorbed by the PCM 210.

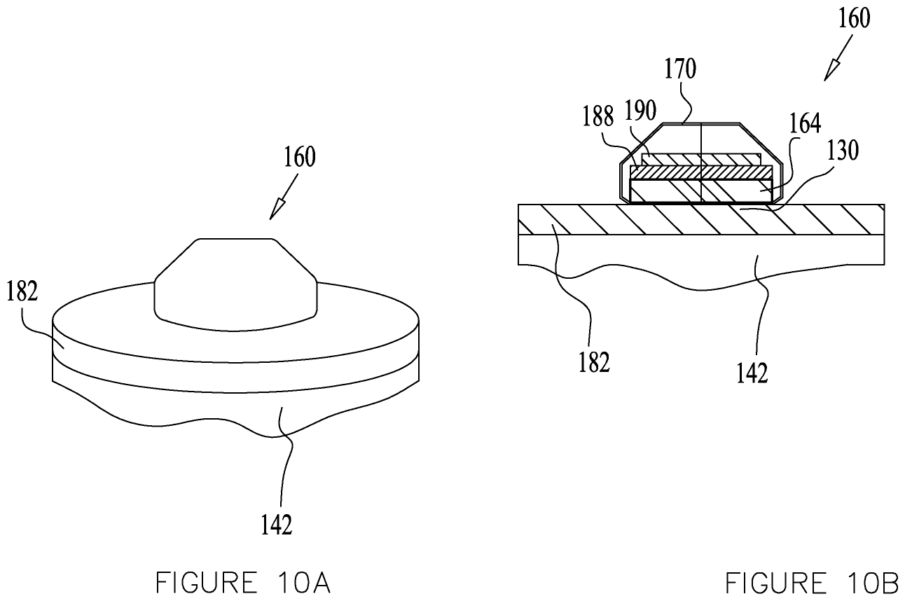

As seen in FIGS. 10A and 10B, in some embodiments, the treatment apparatus 160 may comprise the cooling element 164, such as the thermoelectric cooler 174 and the battery 188 and controller and electronics 190. Removal of heat may be performed via the housing 170 and/or the adhesive 182.

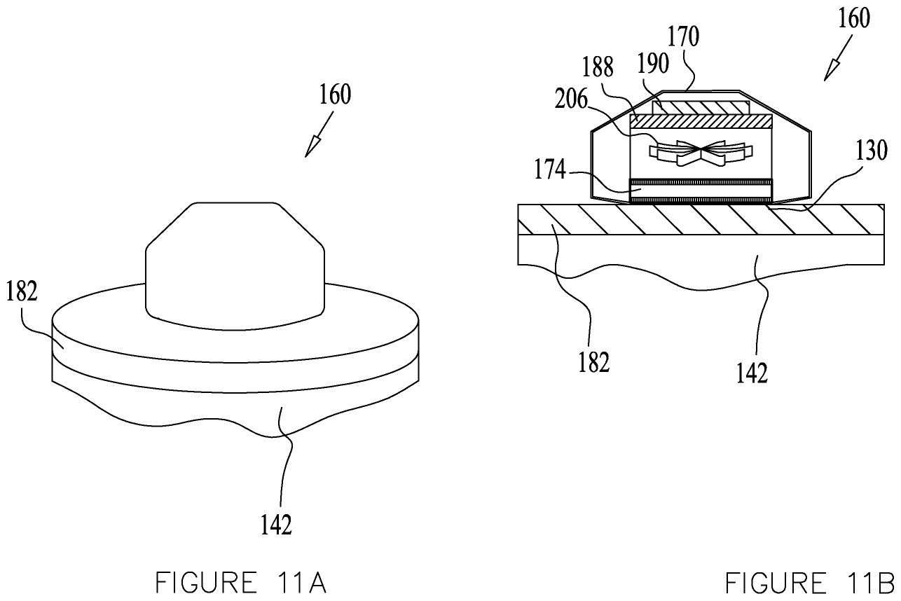

As seen in FIGS. 11A and 11B, in some embodiments, the treatment apparatus 160 may comprise a cooling element 164, such as the thermoelectric cooler 174 and the battery 188 and controller and electronics 190. Removal of heat may be performed by the heat disposal assembly 166 via fan 206 that blows the now heated air, which is ejected from the treatment apparatus 160 via the housing 170 and/or the adhesive 182.

In the embodiment of FIGS. 12A and 12B, the heat disposal assembly 166 may include fans or blowers 206 positioned in any suitable location, at the sides of the treatment apparatus 160 where the removed heat is blown upwardly via apertures 240 formed in the housing 170. Alternatively or additionally, a single or plurality of lateral blowers 206, where the removed heat is blown sideways, may be positioned within the treatment apparatus 160. Fan or blower 206 may be formed as a relatively small fan or microfan or as a micro blower, such as solid state heat blower, for example.

Designing the treatment apparatus 160 for effective cooling and/or heating, using low power supply and for unbulky, small sized and comfortable placement on the body may be challenging. In a non-limiting example, the treatment apparatus 160 may be sized to be relatively small, such as with a volume of about 50 cubic centimeters, or a volume in a range of about 30 to about 100 cubic centimeters, or a range of about 30 to about 60 cubic centimeters or in a range of about 40 to about 55 cubic centimeters or subranges thereof. The required power supply may be low, such about 1 Ampere per hour battery, or a power supply in a range of about 0.5 to about 2 Ampere per hour, in a non-limiting example.

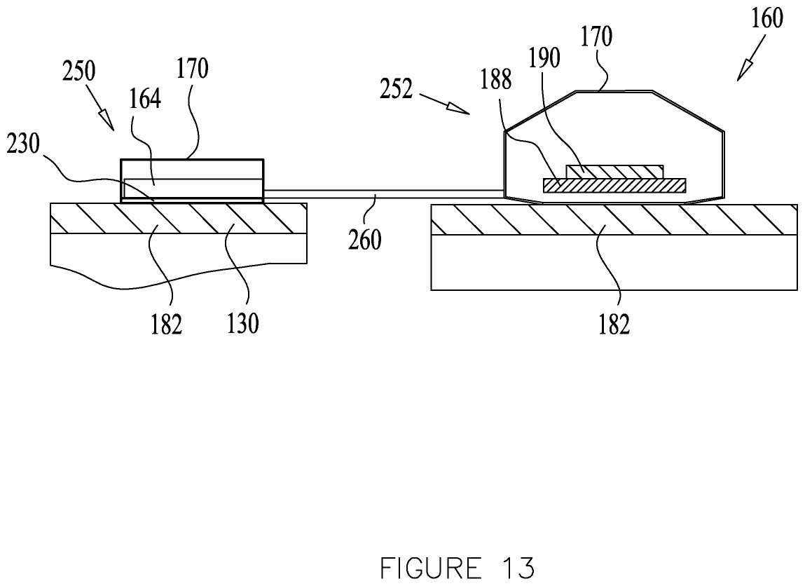

As seen in FIGS. 13-15, the treatment apparatus 160 may comprise a first on-site unit 250, containing minimal components and may be relatively small-sized for cooling the injection site 130, coupled to a second, remote unit 252 housing the remaining treatment device components at a remote location away from the injection site 130.

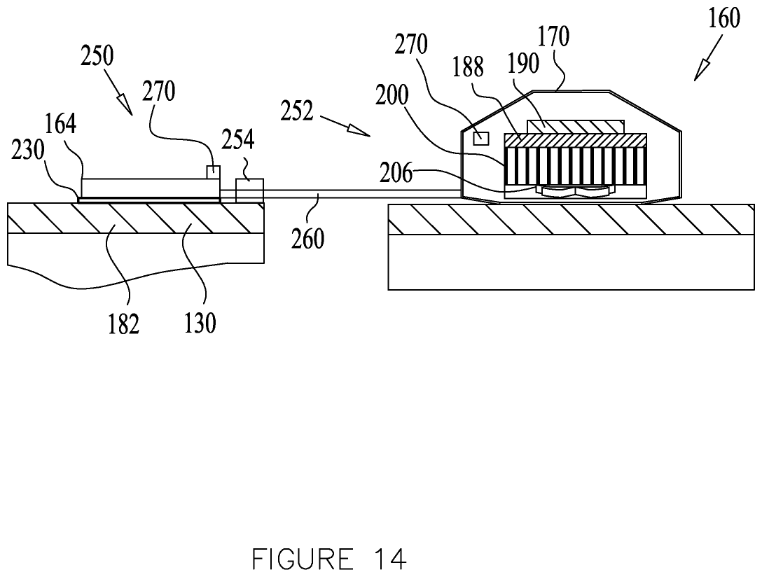

The on-site unit 250 comprises relatively few components, such as a metal plate 230 and an adhesive 182, or the adhesive may be formed as a thermal plate 194 (FIG. 3B). In some embodiments, as shown in FIGS. 13 and 14, the on-site unit 250 may comprise the cooling element 164, such as a resistor or thermoelectric cooler 174. In some embodiments, the on-site unit 250 may just comprise a plate or layer, operative to cool (or heat) the injection site 130.

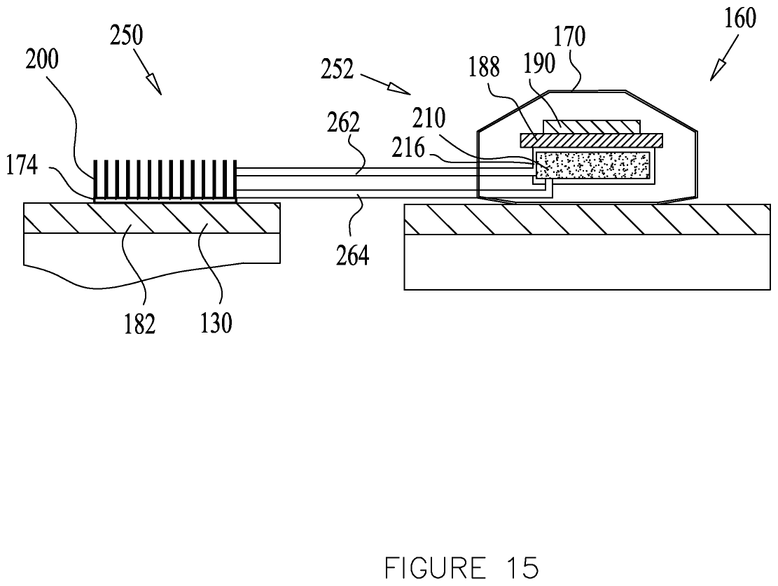

In some embodiments, as seen in FIG. 15, the cooling element 164 may comprise the thermoelectric cooler 174. The heat disposal assembly 166 comprising the heatsink 200 may be provided in conjunction with the thermoelectric cooler 174, and may be arranged above the thermoelectric cooler 174 for dissipating the removed heat to the ambient. The on-site unit 250 may or may not comprise the housing 170.

The remote unit 252 may comprise the housing 170 containing the battery 188 and electronics and controller 190 and any other additional features of the treatment apparatus 160. In some embodiments, as shown in FIG. 14, the remote unit 252 may further comprise the heat disposal assembly 166 such as the heatsink 200 and/or the fan 206 and/or the PCM 210 for dissipation of the heat removed by the on-site unit 250. In some embodiments, as seen in FIG. 15, the remote unit 252 may comprise a heat storage apparatus such as the PCM 210 which may be placed in a vacuum chamber 216 or may at least partially be surrounded by thermal insulation.

The remote unit 252 may be placed away from the injection site 130, such as at a location where the remote unit 252 may be comfortably placed or worn, such as within the user's pocket or latched on a belt or clothing or placed within a pack or bag or other container or adhered to another location on the body. The distance between the on-site unit 250 and the remote unit 252 may be any suitable distance such as from a few centimeters to tens of centimeters to a meter or more.

In some embodiments, the on-site unit 250 may be coupled to the remote unit 252 via at least one conduit 260 or more. The conduit 260 may be configured to direct the removed heat from the on-site unit 250 to the remote unit 252, such as via a transfer fluid, for example. The transfer fluid may comprise a cooling fluid.

In some embodiments, the on-site unit 250 or remote unit 252 may comprise a cooling chamber 254 (FIG. 14) configured for cooling the cooling fluid flowing therethrough from conduit 260.

In some embodiments, the cooling (or heating) may be performed in a closed loop wherein the cooling fluid circulates within the tubes 260. The cooling fluid flows to the injection site in a cold state, thereby cooling the injection site 130. The removed heat may be transferred via the cooling fluid from the on-site unit 250 back to the remote unit 252 for dissipation thereof. The cooling fluid may be cooled by the cooling chamber 254 and/or by the cooling element 164.

As shown in FIG. 15, there may be provided a first conduit 262 configured for injecting a cooling fluid from the remote unit 252 to the heatsink 200 of the on-site unit 250. The cooling fluid absorbs the heat emitted from the heatsink 200 and flows via a second conduit 264 back to the remote unit 252, where the fluid is cooled again by the PCM 210. The cooling fluid may maintain its low temperature by emitting its heat to be absorbed by the PCM 210. The temperature of the cooling fluid may be determined in accordance with the phase transition temperature of the PCM 210.

In some embodiments, the closed loop cooling may be performed by a thermoelectric cooler 174 arranged on the on-site unit 250. The lower plate 176 cools the fluid as it flows to the injection site 130. The hot upper plate 178 is in thermal contact the conduits 260 such that the removed heat is transferred via the cooling fluid to the remote unit 252.

In some embodiments, the conduit 260 may comprise a heat pipe including two solid interfaces. At a hot interface of the heat pipe a liquid may be in contact with a thermally conductive solid surface. The solid surface may comprise the upper plate 178 of a thermoelectric cooler 174 placed on the on-site unit 250 and/or the remote unit 252. The liquid turns into a vapor by absorbing heat from that surface. The vapor then travels along the heat pipe to the cold interface, such as the lower plate 176 and condenses back into a liquid, thereby releasing the latent heat. The liquid then returns to the hot interface through either capillary action, centrifugal force, or gravity, for example.

In some embodiments, the on-site unit 250 and/or the remote unit 252 may be provided with a thermal switch, such as thermal switch 220 of FIG. 8B, for selective thermal communication between the on-site unit 250 and the remote unit 252. The thermal switch 220 may be configured to stop the fluid flow in conduit 260 to deactivate the applied treatment or to start the fluid flow upon activation of the treatment.

In some embodiments, there may be electrical communication between the on-site unit 250 and the remote unit 252, such as via a wired connection for operating the thermoelectric cooler 174 or via a wireless connection comprising transceivers 270 arranged at the on-site unit 250 and the remote unit 252, as shown in FIG. 14. In some embodiments, the on-site unit 250 and the remote unit 252 may be electrically isolated from each other.

In some embodiments, the on-site unit 250 and the remote unit 252 may be thermally isolated from each other and the conduit 260 may be obviated. The controller 190 may activate the cooling element 164 wirelessly or via a wired connection. The removed heat may dissipate to the ambient at the injection site 130, such as via the heatsink 200 and/or via the adhesive 182 or by any other suitable manner.

In some embodiments, the treatment apparatus 160 shown in FIGS. 2A-15 may be used for both cooling and heating and, accordingly, decreasing and increasing the drug absorption in the circulatory system 152. In some embodiments, the treatment apparatus 160 may further comprise a heating element 280 (FIG. 5B) such as a resistor or any other heater for heating the injection site 130. In some embodiments, the thermoelectric cooler 174 may be used to heat the injection site 130 by switching the directionality of the electric current the heat may flow from the upper plate 178 to the bottom plate 176.

In any one of the embodiments of FIGS. 2A-15, an electric current, generated by the temperature difference between the thermoelectric cooler 174 upper plate 178 and bottom plate 176, may be used to charge the battery 188.

In any one of the embodiments of FIGS. 2A-15, a safety mechanism may be provided, such as a thermal switch, e.g. thermal switch 220. The thermal switch 220 may stop the cooling mode whereupon it is detected by the temperature sensor that the injection site 130 is overheating, such as due to failure of the heat disposal assembly 166 to properly direct the removed heat away from the injection site 130.

In any one of the embodiments of FIGS. 2A-15, the treatment apparatus 160 may comprise a charging port for charging the battery 188. Charging may also be utilized for returning the PCM 210, upon inadvertent phase change, back to its solid state.

In some embodiments, the treatment applied by the treatment element 118 can include, but not be limited to, for example, any one of: electrical, magnetic and/or mechanical stimulus, such as a thermo-treatment element for heating and/or cooling; mechanical vibrations, suction, massaging, acoustic stimulation (e.g., ultrasound), electromagnetic radiation, electric field stimulation, magnetic field stimulation, radio frequency irradiation, microwave irradiation, electrical stimulation, magnetic stimulation, Transcutaneous Electrical Nerve Stimulation (“TENS”), or the like, and/or any combination of the above treatments to affect the release rate of the drug from the drug depot 150 into the circulatory system 152. In some embodiments, the treatment element 118 can stimulate or inhibit the subcutaneous tissue 142 by introducing additional substances (in addition to the therapeutic fluid), for example, including, but not limited to, drugs, medicament, chemicals, biologically active bacteria, biologically inactive bacteria or the like or also any combination of the above treatments to affect the release rate of the drug from the drug depot 150 into the circulatory system 152.

In some embodiments, applying treatment may thereby modify the chemical structure and transition rate of the drug. Upon heating the injection site 130 the size of the precipitates or microprecipitates may be altered, urging their dissolve and thus transition into hexamers and possibly to dimers and/or monomers and increasing the release rate of the drug from the drug depot 150 into the circulatory system 152, such as when the drug comprises an insulin glargine. Upon cooling the injection site 130 the transition of precipitates or microprecipitates, into hexamers and from hexamers to dimers and/or monomers, is halted or decreased. At times the cooling reverses the chemical transition of at least some of the hexamers back to precipitates or microprecipitates and the dimers and/or monomers back to hexamers, which, accordingly, halts or decreases the release rate of the drug from the drug depot 150 into the circulatory system 152.

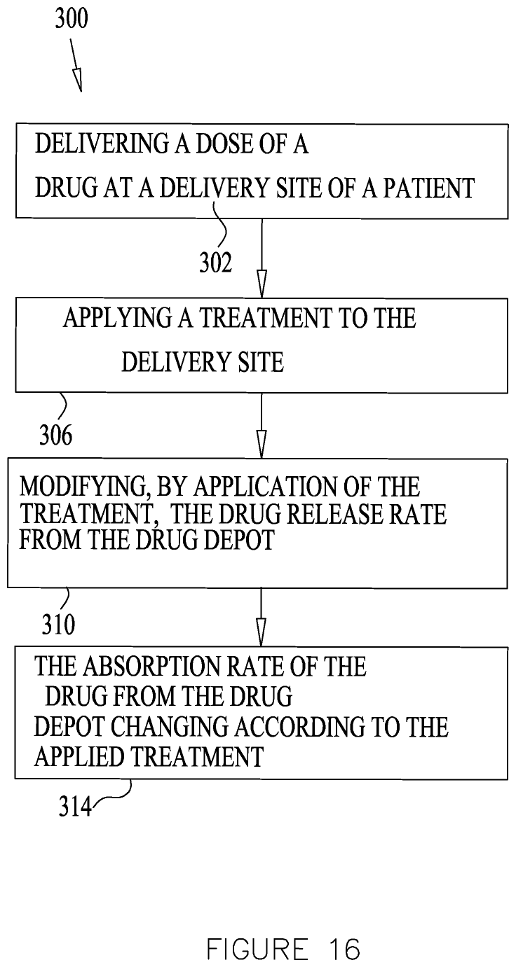

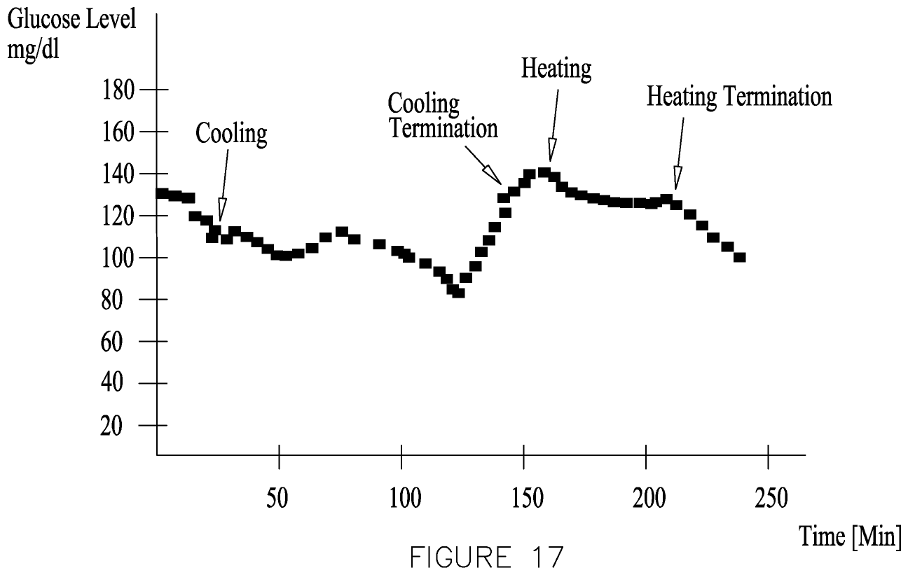

FIG. 16 is an exemplary schematic flow chart of a method 300 for regulating the absorption of a drug in the body of a patient.

A dose of a drug, such as a long acting drug or a short acting drug may be delivered in any suitable manner at the drug delivery site 130 of a patient 302. A treatment (e.g. cooling and/or heating) may be applied to the drug delivery site 130, 306. The treatment may be applied at any suitable time, around the time of the drug delivery and/or unrelated to time the drug was delivered. For example, the treatment may be applied shortly before the drug delivery, a significantly long time before the drug delivery, during the drug delivery, a short time after the drug delivery, and/or a significantly long time after the drug delivery, such at about 1 to about 72 hours, about 1 to about 48 hours, about 1 to about 24 hours after drug delivery, a few hours to 24 hours after drug delivery, about 10 to about 18 hours after drug delivery, about 10 to about 24 hours after drug delivery and subranges thereof. In some embodiments, the treatment is applied a multiplicity of times during the presence of the long acting drug in the drug depot 150.

The drug release rate from the drug depot 150 into the circulatory system 152 may be modified by application of the treatment 310, thereby regulating the absorption of a drug in the body of the patient 314.

The treatment may be applied in accordance with one of the following non-limiting exemplary protocols for regulating the analyte, e.g. the blood glucose level.

In some embodiments, an exemplary protocol may comprise an algorithm configured to predict future deviations of the analyte from the predetermined range by recognizing a trend indicative of a forthcoming deviation from the predetermined analyte range.

In some embodiments, a trend-based algorithm comprises a rate of change of the analyte level.

The rate of change can be calculated is any suitable manner, e.g. as a linear regression of the detected analyte level over a period of time. In this algorithm a current detected analyte level, denoted by Analyte_{now}, is detected at a current time t_{Current}, as well as a previous analyte level Analyteₚᵣₑᵥᵢₒᵤₛ, detected at an earlier time tₚᵣₑᵥᵢₒᵤₛ. Accordingly, the analyte level change rate is calculated as:

$$\frac{Δ Analyte}{Δ time} = \frac{{Analyte}_{now} - {Analyte}_{previous}}{{t}_{now} - {t}_{previous}}$$

As described above, the predetermined analyte level range comprises a lower threshold, here denoted by Analyte_{lower} and an upper threshold, here denoted by Analyteᵤₚₚₑᵣ.

The algorithm may follow the following rules, assuming the time is calculated in minutes, though any other time frame may be considered:

$$If {Analyte}_{now} + \frac{Δ Analyte}{Δ time} \times 60 \leq {Analyte}_{lower} = > Activate cooling mode .$$

The cooling may be deactivated whereupon a normal, predetermined analyte level (also referred to as a second predetermined threshold) and denoted by Analyte_{normal follwing cooling}, is reached such that:

$$Once {Analyte}_{now} + \frac{Δ Analyte}{Δ time} \times 60 \leq {Analyte}_{normal follwing cooling} = > Stop cooling mode .$$

The algorithm may further comprise the additional rules pertaining to the heating mode:

$$If {Analyte}_{now} + \frac{Δ Analyte}{Δ time} \times 60 \geq {Analyte}_{upper} = > Activate heating mode .$$

The heating may be deactivated whereupon a normal, predetermined analyte level (also referred to as a second predetermined threshold) and denoted by Analyte_{normal follwing heating}, is reached such that:

$$If {Analyte}_{now} + \frac{Δ Analyte}{Δ time} \times 60 \leq {Analyte}_{normal following heat} = > Stop heating mode .$$

Consequentially:

$$If \text{:} {Analyte}_{lower} \leq {Analyte}_{now} + \frac{Δ Analyte}{Δ time} \times 60 \leq {Analyte}_{upper} = > No treatment$$

It is noted that in some embodiments the deactivation of the cooling or heating mode may be performed upon passage of a predetermined time period, such as a few minutes to a few hours.

It is noted that the lower threshold, Analyte_{lower} and the upper threshold, Analyteᵤₚₚₑᵣ may be selected to be any suitable predetermined analyte value.

In a non-limiting example for the above algorithm based on the analyte level trend, the analyte may be the blood glucose (BG) monitored by the blood glucose monitoring (BGM) sensor (e.g. by biosensor 102). The long acting drug may be an insulin-containing drug. The Δtime may be calculated in minutes.

The predetermined range may comprise the lower threshold, BG_{lower}=70 mg/dl and the upper threshold, BGᵤₚₚₑᵣ=160 mg/dl.

$$\frac{Δ BG}{Δ time} = \frac{{BG}_{now} - {BG}_{previous}}{{t}_{now} - {t}_{previous}}$$

The cooling may be activated upon:

$$If {BG}_{now} + \frac{Δ BG}{Δ time} \times 60 \leq 70 mg \text{/} dl = > Activate cooling .$$

The cooling may be deactivated whereupon a normal, predetermined blood glucose level is reached. In an non-limiting example this blood glucose level is determined to be 140 mg/dl such that:

$$Once {BG}_{now} + \frac{Δ BG}{Δ time} \times 60 \geq 140 mg \text{/} dl = > Stop cooling .$$

The algorithm may further comprise the additional rules:

$$If {BG}_{now} = \frac{Δ BG}{Δ time} \times 60 \geq 160 mg \text{/} dl = > Activate heating .$$

The heating may be deactivated whereupon a normal, predetermined blood glucose level is reached. In an non-limiting example this blood glucose level is determined to be 120 mg/dl such that:

$$Once {BG}_{now} + \frac{Δ BG}{Δ time} \times 60 \leq 120 mg \text{/} dl = > Stop cooling .$$

In some embodiments, a data processing filter may be utilized so as to filter undesired noises from the detected blood glucose level. An exemplary filter may be the linear quadratic estimation (LQE) filter or any other method for filtering inaccuracies appearing in data measured over time.

FIG. 17 is a graph depicting changes in the blood glucose levels over time measured in minutes of a patient subjected to cooling and heating treatment. The graph illustrates the treatment protocol applied to a patient in accordance with the above algorithm based on the analyte level trend.

The first 25 minutes were without intervention. At the 25th min it was detected that:

BG_{now}=110 mg/dl at t_{now}=25 minutes;

at tₚᵣₑᵥᵢₒᵤₛ=0, BGₚᵣₑᵥᵢₒᵤₛ=130 mg/dl.

Accordingly:

$$110 + \frac{( 110 - 130 )}{25} \times 60 = 62 mg \text{/} dl .$$

This is <70 mg/dl thus cooling was activated.

After cooling for 120 minutes it was detected that BG_{now}=130 mg/dl at t_{now}=145 minutes;

while tₚᵣₑᵥᵢₒᵤₛ=25 minutes, BGₚᵣₑᵥᵢₒᵤₛ=110 mg/dl.

Accordingly:

$$130 + \frac{( 130 - 110 )}{145 - 25} \times 60 = 140 mg \text{/} dl .$$

This is ≥140 mg/dl. Thus the cooling was stopped.

At the 155th minute it was detected that:

BG_{now}=140 mg/dl at t_{now}=155 minutes;

and tₚᵣₑᵥᵢₒᵤₛ=145, BGₚᵣₑᵥᵢₒᵤₛ=130 mg/dl.

Accordingly:

$$140 + \frac{140 - 110}{155 - 145} \times 60 = 2000 mg \text{/} dl .$$

This is >160 mg/dl thus heating was activated. After heating for 55 minutes it was detected that BG_{now}=130 mg/dl at t_{now}=210 minutes;

And tₚᵣₑᵥᵢₒᵤₛ=155 minutes, BGₚᵣₑᵥᵢₒᵤₛ=140 mg/dl.

Accordingly:

$$130 + \frac{130 - 140}{210 - 155} \times 60 = 119 mg \text{/} dl .$$

This is <120 mg/dl. Thus the heating was stopped.

In the above example the cooling was applied to cool the skin at the drug delivery site 130 to 14° C. and heating was applied to heat the skin at the drug delivery site 130 to 40° C.

It other embodiments, the cooling and heating temperature may be set to any suitable temperature. In another embodiment, the cooling or heating temperature may be determined according to the detected analyte level and/or according to the analyte change rate.

In some embodiments, the trend is a rate of the averaged change of the analyte levels.

In some embodiments, the above trend-based algorithm may comprise calculating the above change rate of the analyte level:

$$\frac{Δ Analyte}{Δ time}$$

in accordance with a weighted sum model. In this model the rates of change at a recent time period (namely, in proximity to are given more weight than changes occurring at previous times. This may result in a more accurate indication of the trend or progression of the analyte level reflecting the current analyte change rates.

Accordingly, the analyte level change rate is calculated as:

$$\sum \frac{Δ Analyte}{Δ time} \times f ( {t}_{now - t 1} )$$

Wherein f(t_{now-t1}) is a function that provides the larger numeric significance to a recent detected time, (i.e. closer to (t_{now}).

In a non-limiting simplified example, an analyte change rate is detected over an hour at four separate fifteen-minute increments. The weight is given to each analyte change rate in a receding order, e.g. the most recent detected analyte change rate at is weighted at 40%. The weight given to the preceding detected analyte change rate is 30%. The weight given to the further preceding detected analyte change rate is 20% and the weight given to the first detected analyte change rate is 10%. Accordingly, the analyte level change rate is calculated as:

$$\sum \frac{Δ Analyte}{Δ time} \times f ( {t}_{now - t 1} ) = \sum \frac{Δ Analyte}{60 - 45 min} \times 40 \% + \frac{Δ Analyte}{45 - 30 min} \times 30 \% + \frac{Δ Analyte}{30 - 15 min} \times 20 \% + \frac{Δ Analyte}{15 - 0 min} \times 10 \%$$

In some embodiments, a trend based algorithm may comprise monitoring the analyte change rate from the commencement of the treatment activation, e.g. from the commencement of the cooling and/or heating application. Should it be found that the treatment is ineffective and the analyte level is not regulated then the an alert may be generated via the treatment apparatus 160 or via the user interface 119. Alternatively or additionally, the controller 110 may be operative to correct the cooling or heating parameters, such as the temperature, treatment duration or value.

In some embodiments, the trend may be based on clinical data or the patient's history stored in the memory module 112 of the system controller 110 or in communication therewith.

It has been found that the transition rate from the initial microprecipitates to the hexamers and then to the dimers and/or monomers is largely dependent on a temperature differential in the drug depot such that the larger the temperature differential the faster the transition rate. It has been further found that the release rate of the drug from the drug depot 150 (in the monomer form) to the circulatory system 152 is dependent on the temperature, such that as the temperature is raised, the release rate increases, and as the temperature is lowered, the release rate decreases.

As has been described, it has been found that release of the drug from the drug depot 150 to the circulatory system 152 generally occurs within the “effective temperature range.” In some embodiments, the long acting insulin is composed to be predominantly released from the drug depot at an effective temperature range comprising about 30° C. to about 42° C.

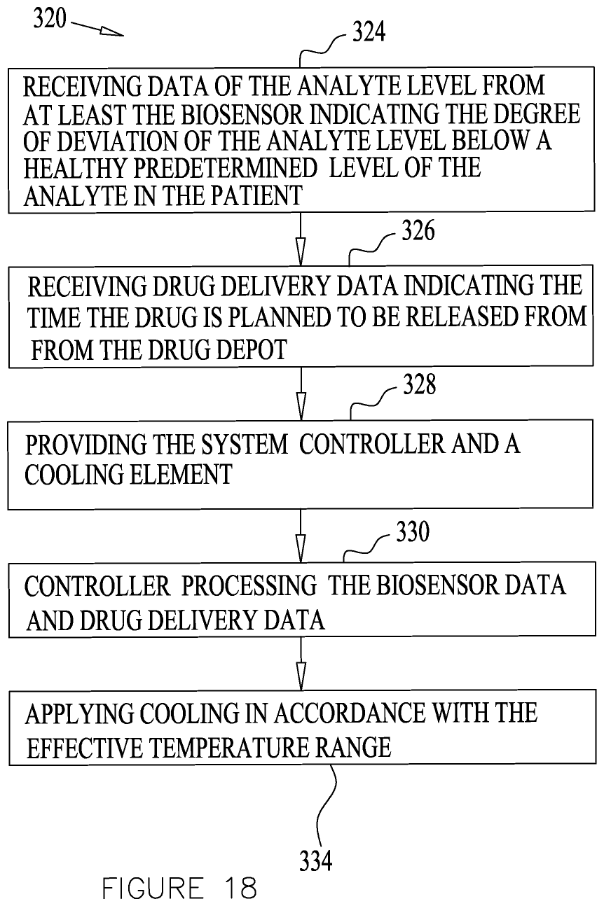

Turning to FIG. 18, there is described a method 320 for reducing the release rate of a drug, e.g. the long acting insulin, from a patient drug depot 150 into a patient circulatory system 152. In this method 320 the treatment is determined in accordance with the effective temperature range as well as additional bodily-function related input.

The method comprises receiving the analyte level data from at least the biosensor 102 indicating the degree of deviation of the analyte level below the healthy, predetermined temperature range or level 324. For example, the biosensor 102 comprises a blood glucose meter and the analyte level is the blood glucose level in the patient.

Drug delivery data may be received, the data indicating the time the drug is planned to be released from the drug depot 150 into the circulatory system 152, 326. The drug delivery data may be calculated by the system controller 110 and may be based upon data received via the user interface 119. For example, the user may indicate that a meal will be consumed at a certain hour, thus indicating that there is a need for insulin release due to an expected rise in blood glucose levels caused by the meal consumption. For another example, the drug delivery data may be received via the bodily function sensor 157 detecting an activity level of the patient, such as exercising indicating the need for insulin decrease due to an expected drop in blood glucose levels following physical activity. For another example, the drug delivery data may be received via the bodily function sensor 157 detecting an activity level of the patient, such as sleep, indicating there may be no need for insulin decrease or increase due to no expectation of rising or drop in blood glucose during slumber hours.

The system controller 110 and cooling element 164 may be provided 328, as described in reference to FIGS. 1A-15. The cooling element 164 may be configured for cooling the drug delivery site 130 to a temperature lower than the effective temperature range (e.g. lower than about 30° C. to about 42° C.) The cooling may be operative to reduce the release rate of the drug from the drug depot 150 into the circulatory system 152.

The controller 110 may process the biosensor data and drug delivery data 330. Based on the processed data, the controller 110 is configured to determine a cooling temperature and cooling duration and time to commence the cooling required for reducing the release rate of the drug from the patient drug depot 150 so as to raise the analyte level back to the healthy, predetermined level in the patient.

The cooling (and/or heating) may be applied in accordance with the effective temperature range 334.

If it is determined that the time the drug is planned to be released from the drug depot 150 is relatively soon (such as due to an anticipated meal) then the cooling temperature decrease from the predetermined temperature range and the cooling duration will be selected to be relatively small.

For example, if according to the user interface 119 it is indicated that a meal will be consumed during the next hour there is a need to raise the insulin release rate within an hour. Accordingly, the controller 110 will activate cooling at a relatively small temperature decrease from the effective temperature range, such as 28° C., which is a small decrease from the effective temperature range of 30° C.-42° C. The cooling duration will be selected to be relatively short, such as for 20 minutes. This allows for raising the blood glucose level back to the healthy range, yet still allows preparing for the anticipated required insulin release due to the planned meal consumption.

If it is determined that the time the drug is planned to be released from the drug depot 150 is relatively later, then the cooling temperature decrease from the effective temperature range and the cooling duration can be relatively large. For example, if it is determined via the bodily function sensor 157 that the patient is sleeping and thus a meal will not be consumed for a few hours, there is no need to raise the insulin release rate soon. Accordingly, the controller 110 will activate cooling at a relatively large temperature decrease from the effective temperature range, such as down to 15° C., which is a relatively large temperature decrease from the lower threshold of the effective temperature range of about 30° C.-42° C. The cooling duration will be selected to be relatively long, such as for 2 hours.

Performing the method 320 for reducing the release rate of a drug allows for activating the cooling and thus controlling the drug release rate while considering substantially many of the factors which affect fluctuations in the blood glucose level, such as physical activity, sleep and meal consumption, for example, as well as the effective temperature range.

In some embodiments, the system controller 110 may further base the treatment protocol on, inter alia, the detected temperature at the injection site 130 within the drug depot 150 or on the skin surface 128. This temperature may be indicative of the chemical state or structure of the drug within the drug depot. Namely, a lower detected temperature less than the “effective temperature range” (e.g. less than 30° C.) indicates that most of the drug is present in the drug depot in the form of microprecipitates. Should a detected temperature be within the “effective temperature range” in proximity to the lower threshold (e.g. above, yet close to 30° C., such as between 30° C.-35° C.) it may indicate that a large portion remains in the microprecipitate state, yet a large portion transitioned into the hexamer state. Should a detected temperature be within the “effective temperature range” in proximity to the upper threshold (e.g. above, yet close to 42° C., such as between 36° C.-42° C.) it may indicate that a large portion has transitioned into the dimer and/or monomer state.

In some embodiments, the system controller 110 may further base the treatment protocol on, inter alia, the detected ambient temperature. The ambient temperature and/or the injection site temperature may affect the blood perfusion rate at the injection site 130 from the drug depot 150 into the circulatory system 152.

A further exemplary treatment protocol may include detecting a beginning of a meal or anticipation of a meal in the next time frame, e.g. an hour. Accordingly, the treatment element may be activated for a predetermined duration, such as an hour, for example.

Another exemplary treatment protocol may include detecting a blood glucose level between 120-160 mg/dl two hours after a meal. The treatment may be activated for one cycle, such as for a predetermined time period, manually or through an Application (such as an Application operating on a user interface 119 or any other computing device 350 (FIG. 1A)) that will take into account activity level, food intake, time of day, and previous blood glucose levels in the same day or profile of blood glucose levels in previous days.

Another exemplary treatment protocol may include activating the cooling mode upon detecting a blood glucose level of less than 90 mg/dl and trending down.

Yet another exemplary treatment protocol may include, upon glucose measurement of less than 100 mg/dl and trending strongly down, activating the cooling mode. Trending strongly down may be defined in any suitable manner such as a decreasing blood glucose level at a predetermined rate.

Still another exemplary treatment protocol may include, upon anticipating low glucose levels below 70 mg/dl within the next hour, activating the cooling mode.

A further exemplary treatment protocol may include applying the treatment in accordance with preprogrammed models that correspond to daily activities. For example there may be a “meal model” which may activate the drug release from the drug depot 150 (e.g. by heating) for a predetermined time prior and/or during and/or after a meal. There may be an “activity model,” which may activate the drug release inhibitor (e.g. by cooling) for a predetermined time period prior, during, and/or following increased physical activity. There may be a “sleep model” programmed to increase monitoring of blood glucose level drops and accordingly release or inhibit the long acting drug, thereby preventing the risk of nocturnal hypoglycemia or hyperglycemia. The models may be activated by the Application and/or any other suitable device (e.g. device 350 or system controller 110, FIG. 1A) upon receipt of signals indicating the appropriate model, such as a time of day or detection of a meal or activity level, for example. The signal may be provided automatically via an appropriate device or may be entered by the user. For example, the user upon going to sleep may activate the “sleep model” or the “sleep model” may automatically be activated upon reaching a predetermined time.

The system controller 110 may apply a treatment protocol based on, inter alia, any one of the biosensor 102, bodily-function sensors 157 and/or received nutrition information. The treatment protocols may be designed according to the type of drug, patient measurements, statistics, and/or any other suitable factor.

The system controller 110 may further base the treatment protocol on, inter alia, data pertaining to history of the specific user, such as his trends of blood glucose readings until this point in time, history of other days (of the specific user), nutrition, and information related to insulin injections for meal and bolus injections.

The system controller 110 may further base the treatment protocol on, inter alia, data pertaining to a predetermined, fixed passage of time from the latest consumed meal, e.g., heating is applied 10 minutes following consumption of a meal and cooling will be applied 70 minutes after consumption of the meal.

In some embodiments, the system controller 110 may be configured to base the treatment application (and anticipation of deviation from the predetermined analyte level or range) inter alia on medical data and research, for example utilizing a medically known blood glucose level that can project the onset of hypoglycemia.

In some embodiments, the system controller 110 may be configured to specialize the treatment application in accordance with the characteristics of the specific user. For example, the user's personal history and reaction to the drug or to nutrient intake and/or physical activity, as well as personal data (e.g. age, gender etc.)

The system controller 110 may further base the treatment protocol on, inter alia, data pertaining to time past from the last meal, time past from previous long acting drug delivery and/or its dose and/or the drug composition. The system controller 110 may further base the treatment protocol on, inter alia, data pertaining to time past from previous short acting drug delivery, or bolus injection and/or its dose and/or the drug composition.

In a further example, a user with a cardiovascular disease may react differently to the treatment of the treatment element 118 and/or the drug than another user. For example the absorption rate of the drug from the drug depot 130 into the circulatory system 152 may be relatively longer than a non-cardiovascular user since the cardiovascular user's capillaries malfunction. Accordingly, the system controller 110 may activate the treatment, such as cooling, for example, for a longer duration than for a non-cardiovascular user.

The system controller 110 may be configured to self-learn the pattern, trends, reactions and characteristics of the specific user and accordingly specialize the activation of the treatment element 118. This self-learning process may be performed in any suitable method, such as by receipt and analysis of past reactions of the specific user by an App for treatment by treatment element 118, for example, or based on known medical data pertaining specifically to the cardiovascular user (or any other specific user group).

In a non-limiting example, the system 100 may comprise a system for prevention of hypoglycemia.

Utilizing methods and treatment protocols or the system 100 described herein may provide for a relatively quick and simple system for real-time control of the blood glucose level and maintenance of a healthy, balanced analyte level.

Furthermore, for patients that are subjected to short acting drugs, such as bolus insulin injections (along with basal injections or without basal injections), utilizing the system 100 may reduce the number of required bolus injections around (before, during, or after) meals. This is because the system 100 provides for stabilizing the blood glucose level by application of the treatment 118, without requiring further insulin injections.

Moreover, utilizing system 100 allows for control of the blood glucose level without requiring further pharmaceuticals, rather by mechanically or thermally treating the drug delivery site 130.

It is appreciated that the system 100 including the treatment element 118 may be used to decrease the delivery rate of any drug, including drugs other than insulin, e.g. drugs for hypertension or others.

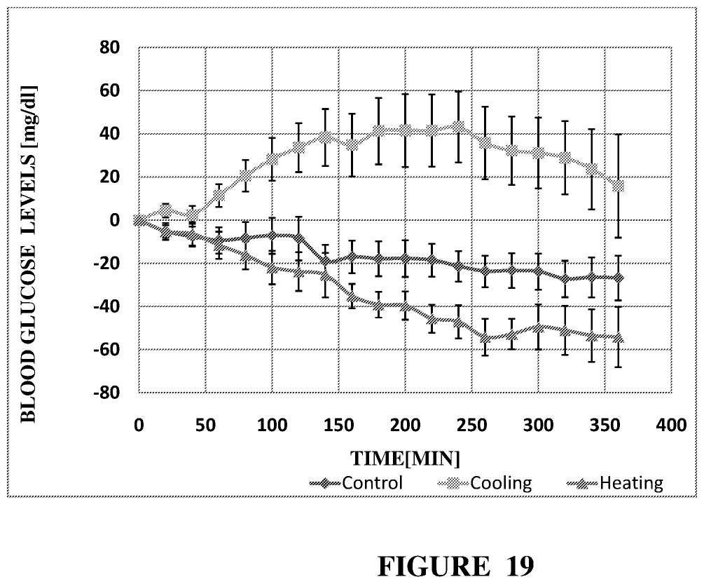

FIG. 19 is a graph showing results of a study of the effect of applying a treatment (cooling or heating) for regulating the absorption of a drug (insulin) on an analyte level (insulin and/or blood glucose level) over time measured in minutes.

### Experiment Procedure

The experiment tests the effect of treatment (e.g. cooling and heating) at the injection site on insulin and glucose blood levels during nine hours following long acting, basal insulin (LANTUS) injection. The tested treatments included heating the skin over the injection site to 40° C. and cooling the skin over the injection site to 20° C. Each subject underwent three procedures during three different days including control, heating and cooling in random order, thus each subject served as his/her own control.

Type I diabetes subjects under fasting conditions injected their usual basal insulin dose which is intended to keep their blood glucose level stable. Two hours after injection a stabilization period commenced in which a subject's blood glucose levels was adjusted to a target blood glucose level using IV insulin or IV glucose administration. The starting glucose level was 150 mg/dl±20 mg/dl. After a stable blood glucose level was observed for half an hour and no interventions (e.g. insulin administration) were performed in that half an hour time frame, the treatment procedure was applied. In the two testing days the heating or cooling elements were attached to the injection site and heating or cooling was started for a period of four hours and no intervention (besides the treatment, i.e. cooling or heating) was performed during this period. The procedure continued for another two hours without intervention. On the control day no intervention or treatment was applied. During the whole procedure blood samples were collected for blood glucose and insulin concentration measurements every 20 minutes. Safety limits of the study were 75 mg/dl as the low limit, and 250 mg/dl as the high limit. If the subject's blood glucose level was below 75 mg/dl, then a 10% glucose solution was given intravenously to increase the subject's blood glucose level. If the subject's blood glucose level was above 250 mg/dl then IV insulin was given to reduce blood glucose level.

### Experiment Results and Analysis

As seen in the graph of FIG. 19 the blood glucose levels are stable around the base line (middle, diamond line) when no intervention was applied to the injection site. Upon heating, blood glucose levels decreased (lower, triangle line) and upon cooling blood glucose levels were increased (upper, squared line).

Table 1 shows blood glucose excursion changes (ABG) from control following cooling or heating after 2, 3 and 4 hours. All results are statistically significant

TABLE 1

| | Cooling | Heating |
|---|---|---|
| ΔBG_2H [mg/dl] | +30 | −29 |
| ΔBG_3H [mg/dl] | +42 | −32 |
| ΔBG_4H [mg/dl] | +50 | −32 |

On average applying heating to the injection site resulted in a decrease of 15, 21 and 26 mg/dl compared to no intervention after 2, 3 and 4 hours respectively. These changes indicate that heating the injection site of basal insulin can result in a reduction of post meal glucose excursion which is clinically meaningful. Applying cooling to the injection site resulted in an increase of about 42, 59 and 65 mg/dl compared to no intervention after 2, 3 and 4 hours respectively.

The observed changes show that the basal insulin injection site treatment technology can be used to control blood glucose levels through changes in the release rate of insulin from the insulin glargine drug depot. There are several benefits, inter alia, using this technology with or without the use of a continuous glucose monitoring (CGM) sensor.

The technology can be used to improve adjustment of the basal rate during the day giving patients on injection therapy the flexible basal rate benefit that pump users enjoy. This feature may keep patients on insulin injection therapy from switching to insulin pump therapy by providing a variable basal rate for those patients.

Applying heating and cooling to the insulin glargine injection site was followed by a decrease and increase in blood glucose levels, respectively. This indicates that heating and cooling of the injection site can increase or decrease the rate of insulin release from the subcutaneous drug depot into the blood stream. On average applying heat to the injection site resulted in a decrease of 40 mg/dl compared to no treatment at all. Heating the injection site may reduce post meal glucose levels by half. In addition, applying cooling to the injection site resulted in a 25 mg/dl increase in blood glucose levels. Extrapolating these results to low blood glucose levels suggests a positive effect and suggests that this technology can be used in cases such as increasing blood glucose levels from 60 mg/dl to 85 mg/dl.

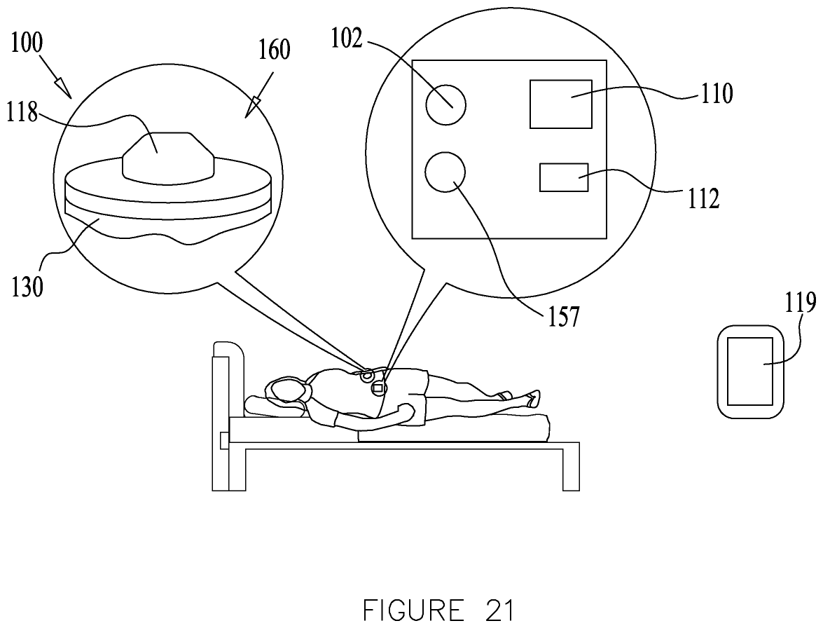

Cooling or heating the insulin depot site (or any other suitable treatment) during the night can be used to reduce the rates of nocturnal hypoglycemic and hyperglycemic events, as described herein and in reference to FIG. 21.

Using the heating application around meal times enables patients on basal insulin therapy to increase the amount of available insulin toward meals, which can result in lower postprandial glucose levels. In a non-limiting example, assuming a reduction of a blood glucose level of 32 mg/dl during 3-4 hours post meal, the HbA1c (glycated hemoglobin) level can be reduced by 0.35%-0.45%.

Cooling or heating the insulin depot site (or any other suitable treatment) enables more efficient and more consistent subcutaneous drug delivery. Implementation of this technology with mealtime insulin enabled multiple daily insulin (MDI) patients to significantly improve their clinical outcome.

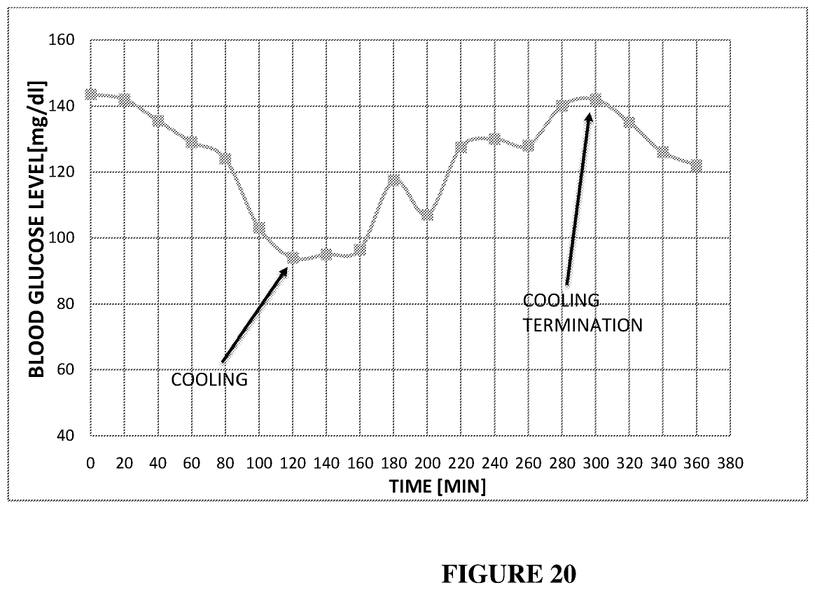

FIG. 20 is a graph showing results of a study of the effect of applying cooling for regulating the absorption of a drug (insulin) on an analyte level (insulin and/or blood glucose level) over time measured in minutes.

### Experiment Procedure

The diabetes subjects under fasting conditions injected their usual basal insulin dose (LANTUS) which is intended to keep their blood glucose level stable. Within 2 hours the blood glucose level decreased from 143 mg/dl to 94 mg/dl. Cooling commenced at 2 hours and was applied for 3 hours successfully raising he blood glucose level back to 142 mg/dl.

### Experiment Results and Analysis

As seen in the graph of FIG. 20, following a sharp trend downwards from the initial blood glucose level of 143 mg/dl during the first 2 hours, the applied cooling successfully raised the blood glucose level back to the initial blood glucose level. This demonstrates that cooling the injection site is effective in controlling the blood glucose level without any other intervention.

As described above, managing illnesses, particularly chronic illnesses, requires monitoring at all times. Yet during times the patient is unaware, such as while sleeping or for patients that are unable to proactively manage their condition, such as children, precautionary measures must be taken to prevent the onset of an emergency associated with the deviation of the monitored parameter (i.e. the analyte) from a normal, healthy level.

As seen in FIG. 21, it is shown that the system 100 can be utilized to prevent deviation from the predetermined analyte level or range at times the patient is unaware or cannot take measures for self-treatment. The system 100 may be configured as a closed loop system wherein the biosensor 102 is configured to be engaged with the patient, here shown in a sleeping state. The biosensor 102 may be conditioned to automatically monitor a certain bodily analyte, without the patient intervention, such as continuously or at predetermined intervals. The detected analyte level is transmitted to the system controller 110 by any suitable means. The system controller 110 may be programmed to determine the deviation of the analyte from the predetermined analyte range as described throughout the disclosure and in reference to FIG. 1A. The predetermined analyte range may be based on known clinical data and/or personal data typical of the individual patient. In some embodiments, an algorithm may be configured to predict future deviation of the analyte from the predetermined range by recognizing a trend indicative of a forthcoming deviation from the predetermined analyte range, as described for example in reference to FIG. 17.

Upon detection of the deviation from the predetermined analyte range, the system controller 110 may initiate an alert directed to the patient or any other caregiver, through the user interface 119. Additionally or alternatively, the system controller 110 may automatically activate the treatment element 118 without user intervention in order to prevent a medical emergency, such as hypoglycemia or hyperglycemia. For example, the patient may be subjected to long acting insulin injections such as shown in FIG. 1B and/or to continuous infusion of insulin, generally by an infusion set (namely an insulin pump) such as shown in FIG. 1C. Upon detection of a decreasing blood glucose level, the treatment element 118 may cool the drug delivery site 130 to decrease the absorption of the drug by the patient to prevent 1 hypoglycemia, such as nocturnal hypoglycemia.

In some insulin infusion systems insulin is infused, at times by a pump and at times in a closed loop system in response to the patient blood glucose level measured by the biosensor 102. These standard infusion systems are capable of infusing the insulin when the glucose level is too high or stopping the infusion when the glucose level is too low, yet these standard infusion systems are incapable of decreasing (or increasing) the delivery rate of the insulin once the insulin was already infused into the body. Thus in a case where the patient was infused by insulin and a drop in blood glucose level is detected, the patient has no means to regulate the blood glucose level. The treatment element 118 is able to provide the means to actively raise the blood glucose level to a normal, predetermined healthy range, by cooling the injection site 130, as described herein in reference to FIGS. 1A-15.

In some embodiments, the treatment element 118 along with delivery of a drug, such as a long acting drug, may mimic and replace the closed loop infusion system. The long acting drug is delivered once within a predetermined time interval (e.g. 24 hours, more or less) and the treatment element 118 is provided to regulate the analyte level based on the biosensor input by applying the treatment to decrease or increase the drug delivery rate from the drug depot 150 to the circulatory system 152.

In some embodiments, a patient may be subjected to short acting insulin therapy and may use the treatment apparatus 160 to control the drug absorption rate during the duration the short acting insulin is present in the drug depot 150 (e.g. 1 or 2 hours). During this time window, the patient may control the drug absorption such as by cooling the drug delivery site 130 to decrease the absorption of the drug by the patient. Decreasing drug absorption may be desired when the patient injected the short acting insulin in preparation for meal consumption yet did not end up consuming the meal. To prevent the absorption of the insulin, cooling may be applied.

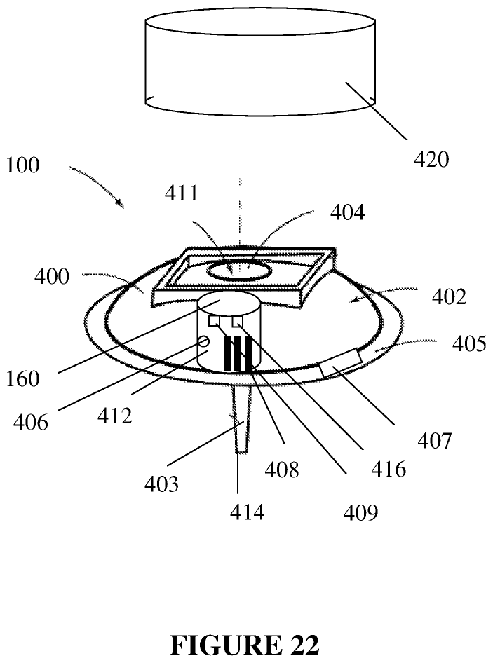

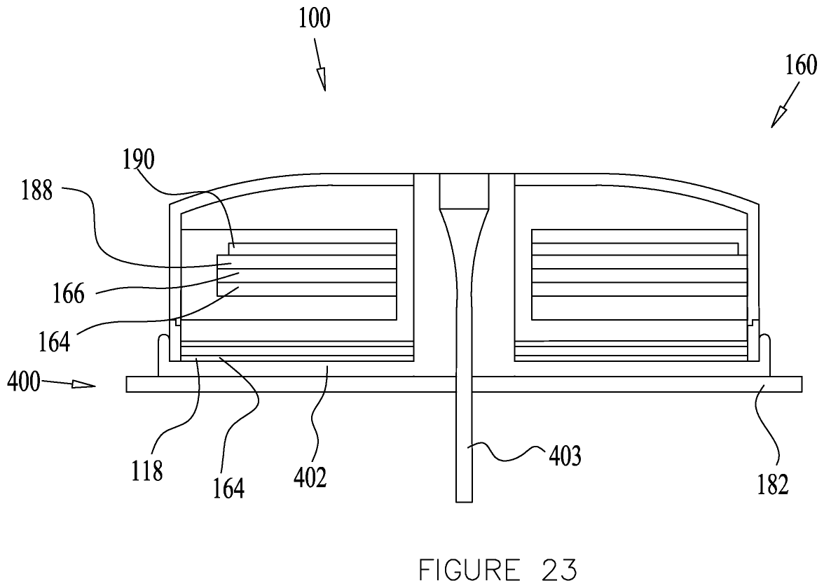

In some embodiments as will be described in reference to FIGS. 22 and 23, the present disclosure relates to systems and methods for delivering drugs to a patient such as by subcutaneous injection of a medicament using an injection port device 400.

Drug injection by syringe, pen-injectors, injection-ports and other devices are used for subcutaneous injections of therapeutic fluids, drugs, proteins, and other compounds for humans or animals. Such delivery systems and methods are used also for insulin delivery. An injection port device is a device adapted for receiving an injection from a syringe or pen injector. The injection port device includes a cannula 403, and may be mountable on a patient with the cannula 403 extending into or through subcutaneous tissue 142 (FIG. 1B) of a patient. For delivering the drug, the syringe with a needle is connected to the injection port device 400 and the drug is injected through the cannula 403 of the injection port to the subcutaneous tissue 142. Thereby, with this injection port device the patient is spared having their skin pierced by the injection needle (e.g. needle 124 in FIG. 1B). After an initial skin piercing by an insertion needle 124 of the injection port device 400, all injections are facilitated via the injection needle being engaged with the injection port device 400 rather than through the skin of the patient. Usually, such an injection port device 400 is replaced every three days.

In many instances, the patient requires insulin injection around the clock to keep proper levels of glucose in the blood. Two major types of insulin can be injected—the long acting insulin, and the short “rapid-acting” insulin, as described herein. In some embodiments, the short “rapid-acting” insulin is injected in relation to meals and provides an amount of insulin for matching a dose of carbohydrates consumed by the patient. When using an injection port device, all types of insulin a patient injects are injected through this port.

When the patient consumes food, his or her levels of glucose rises. Unfortunately, many conventional subcutaneous injection devices, are incapable of quickly matching or preventing the rise of blood glucose. The delay in such matching is also true in case of the “rapid-acting” insulin. Some of the reasons for this delay include a lag in the absorption of insulin from the injection site and the time it takes for complex insulin molecules to break down into monomers

Additionally, since blood glucose levels rise shortly following the meal, the delay in matching insulin to the rising levels causes post prandial hyperglycemic events (i.e., when levels of blood glucose are above normal) to occur. Further, occasionally after a certain period of time passes (e.g., 2-3 hours) after a meal, the blood glucose levels drop yet insulin concentrations in the blood rise followed by the peak of the systemic insulin effect and may result in causing hypoglycemic events (i.e., when levels of blood glucose are below normal) to occur. Further parameters may affect the blood glucose level in the body, such as the activity level, meal type and duration, etc., as described herein, such as in reference to FIGS. 1A-1C and throughout the application. Both hyperglycemic and hypoglycemic events are highly undesirable. Additionally, since blood perfusion rate at the injection site 130 (and hence from the drug depot 150), including that of the injection port device 400, has large variability, depending on the ambient temperature and other parameters, it induces large variations to the delay of the peak of time profile of the insulin action. Those variations in the insulin peak action period further increase the variability in the blood glucose level.

Additionally, it is known that certain drugs including insulin are growth hormones. These drugs when injected several times at the same location can cause local cell growth, causing Lipohypertrophy. Using a regular injected drug being injected several times per day or even during several days at the same location may result in Lipohypertrophy. Increasing local blood perfusion at the injection site promotes drug uptake to the circulatory system, and therefore may reduce this unwanted phenomenon of Lipohypertrophy.

Therefore, it is desirable to provide a system and a method that provides efficient injection and controlled absorption of the drug to the patient circulatory system 152. Controlled absorption of the drug may include where it is desirable to rapidly deliver to the circulatory system 152 or where it is desirable to retard delivery of the drug to the circulatory system. In some embodiments, it is desirable to provide a system and a method for injection of insulin or other drug to the patient through a drug delivery system including an injection port device 400 that: can distinguish between injections of long acting insulin to those of short acting insulin; improves effectiveness of the short acting insulin in the blood to maintain normal levels of blood glucose and prevent or reduce hyperglycemic and hypoglycemic events; and can reduce Lipohypertrophy.

The present disclosure relates to systems, devices and methods for injecting a drug, substances and/or chemicals to a patient that further provides a tissue treatment element for modifying the effectiveness of drug delivery upon injection through the injection port device 400. The treatment is utilized to control and modify drug delivery process by modifying the drug's pharmacokinetic and/or pharmacodynamic profile. In some embodiments, the treatment may come in various forms, for example including an analgesic, vasodilator or the like. In some embodiments, the treatment may be any form of treatment applied by a treatment element 118 that leads to improved vasodilatation of the tissue being injected optionally including but not limited to exposing the tissue region (namely the injection site, such as injection site 130 of FIG. 1A) to energy, radiation, heat, cooling, mechanical vibrations, suction, massaging, acoustic stimulation, electrical stimulation, injection of an additional substance(s), or any combination of the above to modify the drug's pharmacokinetic and/or pharmacodynamic profile. Each treatment type may optionally have a separate protocol in order to evoke the necessary reaction such as vasodilatation or decrease of the drug release rate the like.

In some embodiments, the applied treatment induces vasodilatation through neural stimulation of the tissue around the drug injection site through the injection port device 400. The neural stimulation can be induced by thermal stimulation. The human neural response to thermal stimulation includes several mechanisms such as the Nociceptive Axon Reflex that induce vasodilatation among other effects.

In some embodiments, the induced neural response, such as the nociceptive axon reflex, also optionally induces widening of the capillary pores and increasing the capillary wall permeability. This effect is also significant for improving the absorption of the drug through the capillary wall.

In some embodiments, the applied treatment may lead to a reduction in the variability of the drug absorption in the circulatory system 152 and its local and systemic effects. For example, heating the tissue region in drug delivery site 130 through the injection port device 400 to a preset regulated temperature during and/or after the drug injection and absorption into the blood may cause local blood perfusion at that region to become more reproducible and the drug absorption process may be more uniform and reproducible as well. Also, by reducing the delay between drug injection into the tissue through the injection port device and absorption into the circulatory system, the variability of drug action induced by the delayed profile can be reduced.

In some embodiments, the temperature of the drug depot 150 can be regulated for longer periods, but the cost may be the energy source volume and weight. Therefore, for minimization of the energy source size the heating period or heating temporal profile may be optimized in relation to the period of the drug injection and absorption into the blood. In some embodiments, in which the treatment utilized is for example heat, the drug interaction with the treatment substance or type will be preferably taken into considerations and avoided. For example, a drug's temperature sensitivity will be accounted for so as to avoid protein denaturisation. For example, insulin is a temperature sensitive protein. To avoid damage to the insulin the treatment protocol, for example heat, will be limited so as to ensure the efficacy of the delivered drug. For example, the treatment protocol may control the temperature of the drug delivery site 130 so as to not damage the drug. For instance, heating some types of insulin above an efficacious temperature, such as 37° C., or 39° C., or 40° C. might damage the insulin. So the tissue around the injection site can be heated to induce the required response without heating the insulin itself above the efficacious temperature. For example heating the tissue at a distance of about 10 mm around the injection site 130 to 38.5° C. provides significant vasodilatation without heating the injected insulin above 37° C. It is also possible to heat with a spatial distribution of temperatures around the center of the injection port device in a manner where away from the center the heating is to a higher temperature, while closer to the center the heating is to a lower temperature.

In some embodiments, the applied treatment may lead to a decrease of the drug absorption in the blood or lymph system and its local and systemic effects. For example, such as cooling the injection site, for decreasing the release rate of the basal insulin-containing drug, thereby preventing the further deviation of the glucose level from the normal level.

As seen in FIGS. 22 and 23, the system 100 comprises the injection port device 400 and a treatment apparatus 160. The system 100 may, in some embodiments, includes a disposable part and a reusable part. The disposable portion (or “disposable part”) includes the injection port device 400 comprising an injection port base 402, a cannula 403 extending from the base 402, an optional self-sealing member 404 and an adhesive pad 405 (which may include the adhesive 182 of FIGS. 2A and 2B). The reusable portion (or “reusable part”) 412 may include the treatment apparatus 160 comprising a treatment element 406 (e.g. the treatment element 118), sensors 407, such as a temperature sensor and/or an analyte sensor, electrical components and contacts 408 as well as communication means with a user interface and a power supply. In some embodiments the reusable portion 412 may include mechanical securing elements 409.

The base 402 may include an accessible surface having a single inlet port 411 therein, an engagement surface having a single outlet port 414 at the edge of the cannula 403 and a drug delivery channel extending between the single inlet port 411 and the single outlet port 414. Electrical contacts 408 may be used to facilitate communication between the treatment element 406 and the sensors 407 and the treatment apparatus 160 or any other components in the system 100.

In some embodiments, the reusable portion 412 may include a power supply 188, controller and electronics 190, potentially at least one led indicator 416, and electrical contacts positioned to be in contact with the electrical contacts 408 of the injection port device 400, such as when the reusable element is locked to it's position on the body by the mechanical securing elements 409.

Electronics and the controller on the reusable portion may be used to sense injection of a drug through the inlet port of the injection port device that requires activation of the treatment in order to control the delivery rate of the drug.

In some embodiments, activation of the treatment may be initiated only when needed. This can be achieved by having means to distinguish between injections of drugs that require treatment to injection of drugs that do not require treatment or it is not desirable to have treatment. This can also be activated by the patient that can press a button or perform a different action for different injection types. Automatic recognition can be achieved for example, by having different shapes formed on the injection port device 400 or treatment apparatus 160 fitted to injectors used to inject a drug that requires treatment and other shapes fitted to injectors that inject drugs which do not require treatment. The different shape can apply pressure or force on a part of the injection port device 400 where electrical contacts are included that then trigger the controller to indicate that a drug which requires treatment is injected. Other means to achieve such automatic recognition may include RFID on the different injectors that are sensed by a sensor on the reusable part which then signal the controller regarding different types of injected drugs.

Additional ways to achieve automatic identification of injected drugs to initiate treatment only when it is desirable to apply treatment may include examples outlined below.

Electronics (both active that send signals and receive signals or passive that does not send signals but can be read by other electronics from remote devices such as a user interface 119) in the reusable part of the injection port device that can communicate or identify electronics (both active that send signals and receive signals or passive that does not send signal but can be read by other electronics from remote devices) in the injection syringe which contain identification information on the drug contained in the syringe.

Other mechanical identification means mounted on the injection syringe or part of the injection syringe that are made unique to a syringe according to the drug contained within the syringe. When the syringe is in contact with the injection port device these mechanical means can trigger activation of a treatment as they come in contact with parts of the injection port device 400.

Parts of the injection port device that can be made to rotate or be pressed and the user rotates or applies pressure on them when treatment is desirable.

According to the drug information received by the controller of the reusable part, the temporal and spatial format of the treatment is activated or not activated.

Additionally, in some embodiments, the electronics on the reusable part of the injection port device can receive or sense information contained in electronics on the injector related to the amount of drug to be injected or amount of drug that was actually injected to determine the temporal profile of the treatment.

Additionally in some embodiments, the electronics on the reusable part of the injection port device can detect mechanical sections of the injection syringe that contain information on the amount of drug to be injected to determine further the temporal profile of the treatment.

Additionally in some embodiments, mechanical features on the reusable part or the disposable part of the injection port can detect mechanical sections of the injection syringe when they are in contact with them. These may contain information relating to the amount of drug to be injected to determine further the temporal profile of the treatment.

The injection port can be used for example for at least one day and up to few days of insulin injections. It can be attached to the abdomen or other locations used for insulin injection when the first injection should be taken. The reusable portion can be connected to the disposable portion before or after positioning of the disposable portion on the body, using an insertion unit 420 for example, and securing it to the skin. To inject the drug the patient may use the appropriate syringe and needle, injecting the drug through the injection port device. The controller on the reusable part determines if this drug requires treatment and, if treatment is desired, further determines the spatial and temporal profile of the treatment. A short time before, after, or during the injection, the predetermined treatment profile is initiated. For example, the treatment element apparatus 160 can heat, such as for 30 min to 38.5° C., or cool for a predetermined time to a predetermined temperature. In some cases the patient can manually start the treatment at a longer period before injection, such as fifteen minutes, to maximize the effect. In some cases the controller can have some delay before starting to heat or cool or apply any other treatment.

In some embodiments, the temperature sensor for providing temperature readings to the controller in the reusable part is embedded in the disposable portion with sufficient thermal communication with the treated tissue and/or the treatment element and connected through the electrical components to the reusable part. In some embodiments, to reduce disposable part costs, the temperature sensor is embedded in the reusable part with thermal communication to a heat conductive element disposed in disposable part. This may enable the temperature sensor to measure temperature of the heated or cooled tissue with good enough accuracy in order to control its temperature. In some embodiments, the heat conductive element can be a metal strip, such as Cu or Al disposed at the bottom of disposable sticker with good heat conduction to heated tissue and/or heating element. In some embodiments, the heat conductive element can be a metal strip, such as Cu or Al disposed at the bottom of disposable sticker with good heat removal from the cooled tissue and/or cooling assembly.

In some embodiments, the patient can detach the reusable part from the disposable part, which is kept adhered to the patient body. In this case, a smaller battery may be used to be disposed inside reusable part since it is required to provide the power only for one injection and the overall size and weight of the reusable part can be reduced. In some embodiments, the battery disposed inside reusable part is capable of providing power for treatment of four injections or more than four injections. In some embodiments LED indicators 416 are used for indication of heating operation (green LED on), low battery (flashing yellow LED) and for any fault or misuse indication (yellow LED on).

In some embodiments, the injection port device 400 may comprise the base 402, the cannula 403 and the adhesive pad 405 or any other attachment means. The insertion unit 420 may be used to secure the base 402 to the skin.

Turning to FIG. 23, it is seen that the system 100 may comprise the injection port device 400 engaged with a treatment apparatus 160 configured for cooling, as described in reference to FIGS. 2A-15. The injection port device 400 may comprise the base 402 and the cannula 403 extending therefrom and attached to the skin via the adhesive pad 182. The injection port device 400 may be formed to be placed on the skin for a relatively long duration, such as a few days, and then may be disposed and replaced by a new injection port device 400 thereby rendering the injection port device a disposable portion. Insertion of the injection device port 400 may be formed by the insertion unit 420 (FIG. 22) for inserting the cannula 403 into the tissue and attaching the adhesive pad 182 and base 402 on the skin.

Upon removal of the insertion unit 420 the treatment apparatus 160 may be placed on the base 402 for applying treatment to the tissue. Treatment apparatus 160 may comprise the reusable portion 412. Treatment apparatus 160 may include the power supply 188. The treatment apparatus 160 may comprise the controller and electrical contacts 190 for controlling and activating the treatment element 118 (e.g. the cooling element 164). The treatment apparatus 160 may be formed in any suitable manner such as described herein. In some embodiments the treatment apparatus 160 may be formed with a heater to heat the injection site 130. In some embodiments the treatment apparatus 160 may be formed with the cooling element 164 to cool the injection site 130 and the heat disposal assembly 166 and may be formed as described in reference to FIGS. 2A-15.

Any one of the embodiments described herein in reference to FIGS. 1A-21 may be utilized with injection port device 400 described in reference to FIGS. 22 and 23.

Communication between the biosensor 102, the controller 110, the user interface 119 other devices 350 and any other components of the treatment element 118 or a component of the system 100 can be provided in any suitable manner. In some embodiments, the communication can be wired and provided through electrical connections. In some embodiments, the communication can be wireless via an analog short range communication mode, or a digital communication mode including WIFI or BLUETOOTH®. Additional examples of such communication can include a network. The network can include a local area network (“LAN”), a wide area network (“WAN”), or a global network, for example. The network can be part of, and/or can include any suitable networking system, such as the Internet, for example, and/or an Intranet. Generally, the term “Internet” may refer to the worldwide collection of networks, gateways, routers, and computers that use Transmission Control Protocol/Internet Protocol (“TCP/IP”) and/or other packet based protocols to communicate therebetween.

In some embodiments, the system 100 may comprise a single or plurality of transmission elements for communication between components thereof. In some embodiments, the transmission element can include at least one of the following: a wireless transponder, or a radio-frequency identification (“RFID”) device. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to-digital (“A/D”) converter, and/or an electrical circuit for analog or digital short range communication.

In some embodiments, the controller 110 and/or any other relevant component of the system 100 can include a processor, a memory, a storage device, and an input/output device.

Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof. These various implementations, such as associated with the system 100 and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., non-transitory mediums including, for example, magnetic discs, optical disks, flash memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smartphone, media player or personal data assistant (“PDA”). Other kinds of devices may be used to provide for interaction with a user as well. For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

The components of the system may be interconnected by any form or medium of digital data communication (e.g., a communication network). Examples of communication networks include a local area network (“LAN”), a wide area network (“WAN”), and the Internet. The computing system according to some such embodiments described above may include clients and servers. A client and server are generally remote from each other and typically interact through a communication network. The relationship of client and server arises by virtue of computer programs running on the respective computers and having a client-server relation to each other.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements/features from any other disclosed methods, systems, and devices, including any and all features corresponding to translocation control. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure).

1. A drug delivery control apparatus configured to control an amount of drug contained in a drug depot delivered or otherwise perfused or diffused into the circulatory system of a patient comprising:
a cooling element configured for cooling a treatment area by removing heat from the treatment area, the cooling element to be arranged above or near the treatment area,
a heat disposal assembly in thermal communication with the cooling element and configured for directing the removed heat to a heat zone away from the treatment area;
a power source;
a controller; and
a housing configured to at least partially house at least the cooling element and the heat disposal assembly.

2. A drug delivery control apparatus configured to control an amount of drug contained in a drug depot delivered or otherwise perfused or diffused into the circulatory system of a patient comprising:
a cooling element configured for cooling a treatment area by removing heat from the treatment area, the cooling element to be arranged above or near the treatment area,
a heat disposal assembly in thermal communication with the cooling element and configured for directing the removed heat to a heat zone away from the treatment area;
a power source;
a controller; and
a housing configured to at least partially house at least the cooling element and the heat disposal assembly.

3. The apparatus according to claim 1, wherein
the cooling element comprises a thermoelectric cooler having at least a first plate and a second plate; and
the heat disposal assembly comprises a thermal conducting adhesive configured to direct heat to the heat zone, and a heatsink in thermal contact with the first plate of the thermoelectric cooler.

3. The apparatus of claim 1, wherein the heat disposal assembly comprises a fan.

4. The apparatus of claim 2, wherein the controller is configured to control the thermoelectric cooler to apply heat or cooling to the treatment area.

5. The apparatus of claim 1, wherein the heat zone is spaced away from the treatment area between 2 to 5 centimeters.

6. The apparatus of claim 1, wherein the housing comprises a thermal conductive case.

7. The apparatus of claim 1, wherein the housing includes a plurality of at least one of folds and creases.

8. The apparatus of claim 1, wherein the housing comprises fins forming a radiator-like structure.

9. The apparatus of claim 1, wherein the heat disposal assembly comprises a phase change material configured to absorb at least some heat from the treatment area.

10. The apparatus of claim 1, wherein the treatment area comprises a drug delivery site for delivery and storage of a drug to a drug depot comprising an area within a subcutaneous tissue layer proximate the drug delivery site, and the apparatus is configured to heat or cool the treatment area, such that a change of the local tissue and local circulatory system properties affecting the drug contained within the drug depot, is established.

11. The apparatus of claim 1, wherein the apparatus further comprises at least one sensor configured to determine at least one analyte level, wherein when the analyte level deviates from a predetermined range, the controller activates a treatment protocol to effect heating or cooling of the treatment area.

12. The apparatus of claim 1, wherein:
the cooling element comprises a thermally conductive plate the thermally conductive plate arranged above or adjacent the treatment area,
the heat disposal assembly comprises a phase change material, and
the apparatus further comprising a thermal switch provided between the thermally conductive plate and the phase change material.

13. The apparatus of claim 12, wherein the thermal switch comprises a mechanical pin, wherein the mechanical pin is configured to establish thermal contact between the thermally conductive plate and the phase change material.

14. The apparatus of claim 13, wherein when the mechanical pin establishes thermal contact, the thermally conductive plate cools down.

15. The apparatus of claim 12, wherein the phase change material is contained within thermal insulation.

16. The apparatus of claim 15, wherein the thermal insulation comprises a vacuum.

17. The apparatus of claim 12, wherein the thermal switch comprises an enclosure arranged between the phase change material and the thermally conductive plate.

18. The apparatus of claim 17, wherein the enclosure is at least partially filled with a fluid to selectively limit thermal contact between the phase change material and the thermally conductive plate.

19. The apparatus of claim 12, wherein the controller is configured to increase the temperature of the thermally conductive plate.

20. The apparatus of claim 1, further comprising:
a first unit comprising at least the cooling element, the first unit arranged above or near the treatment area; and
a second unit comprising at least the power source and the controller, the second unit arranged away from the treatment area,
wherein the first unit and the second unit are thermally connected via at least one conduit.

21-34. (canceled)
